(19) United States
(12) Patent Application Publication
(10) Pub. No.: US 2023/0377543 A1
(43) Pub. Date: Nov. 23, 2023

(54) METHOD FOR GENERATING MUSIC WITH BIOFEEDBACK ADAPTATION

(71) Applicant: MINDSET INNOVATKON, INC, Montreal, Québec (CA)

(72) Inventors: Jacob FLOOD, Montreal (CA); David DOYON, Saint-Constant (CA); Xavier SAINT-MLEUX, Saint-Jean-sur-Richelieu (CA)

(21) Appl. No.: 18/030,694

(22) PCT Filed: Oct. 7, 2021
(86) PCT No.: PCT/CA2021/051416

§ 371 Date: Apr. 6, 2023

Related U.S. Application Data
(60) Provisional application No. 63/088,687, filed on Oct. 7, 2020.

(51) Int. Cl. G10H 1/00; A61B 5/38

(52) U.S. Cl. CPC G10H 1/0025; G10H 1/0041; G10H 1/0091; A61B 5/38; G10H 2210/125; G10H 2210/111; G10H 2220/376; G10H 2240/131; G10H 2220/351

(57) ABSTRACT

A method and a system for generating music for an electronic device are provided. The system is configured to generate a first portion of generative music by combining a plurality of audio stems based on a determined first current state vector; measure, with the biosensor, the EEG data while the first portion of generative music is played by the speakers to the user; in response to determining that the current state should be modified to achieve a desired goal state of the user, determine a second set of music parameters for achieving the desired level of focus of the user; generate by the processor and play at the speaker a second portion of generative music characterized by the second set of music parameters to achieve the desired goal state of the user.

### CROSS-REFERENCE TO RELATED APPLICATION

This application claims priority or benefit from U.S. provisional patent application 63/088,687 filed Oct. 7, 2020, which is hereby incorporated herein by reference in its entirety.

### FIELD

The subject matter disclosed generally relates to automated music generation. More specifically, it relates to a method for generating music which is adapted in real-time based on biofeedback.

### BACKGROUND

Adaptive music (also known as dynamic music or interactive music) involves designing a piece of music in such a way that it can be changed in real-time by outside parameters. Adaptive music is often used in video games (e.g., the music becomes more intense when there are more enemies around you).

Generative music is a form of adaptive music. Whereas adaptive music is created by combining short (˜5-10 seconds) musical stems together, generative music is created without musicians in the loop. It can also include taking pieces of “existing songs” into a remix. In generative music, individual notes are generated from a computer, and combined algorithmically to make a full piece. For the purpose of the description provided herein, adaptive and generative music may be considered the same.

WO2019040524A1 addresses generative music, but fails to address the possibilities of generative music for assessing and improving a level of focus (including “low-stress attention” or other similar mental states) during a work session.

### SUMMARY

In accordance with a first aspect, a method is provided for generating music for an electronic device coupled to a server. The method is executable by a processor located on the server. The processor is coupled to: an audio stems database comprising a first plurality of audio stems and a second plurality of audio stems; and a speaker and a biosensor located in the electronic device, the sensor being configured to measure an electroencephalographic (EEG) data of the user, and the speaker being configured to receive and play a generative music.

The method comprises: based on comparing of a first current state vector having a first set of musical parameters with stem label vectors of the audio stems, retrieving a first plurality of audio stems from the audio stem database and generating, by the processor, a first portion of generative music by combining the first plurality of audio stems into a plurality of simultaneously played layers, the first portion of generative music having the first set of musical parameters; measuring, with the biosensor, the EEG data while the first portion of generative music is played by the speakers to the user; determining, by analyzing the EEG data, a second current state vector that characterizes a second current state of the user; based on the determined second current state vector, determining whether a current state should be modified to achieve a desired goal state of the user by determining an error state vector; in response to determining that the current state should be modified, determining a second set of music parameters, for achieving the desired goal state of the user; based on the second set of music parameters, retrieving a second plurality of audio stems from the audio stem database, and combining the second plurality of audio stems to generate a second portion of generative music characterized by the second set of music parameters; and transmitting the second portion of generative music to the speaker and collecting, in real time, a second set of EEG data of the user measured while the second portion of generative music is being played to the user by the speakers.

The processor is coupled to an audio effects database comprising a first plurality of audio effects and wherein generating the first portion of generative music characterized by the first set of musical parameters further comprises combining the first plurality of audio stems with the first plurality of audio effects into a plurality of simultaneously played layers.

In at least one embodiment, determining the first set of musical parameters of the first soundscape is based on a first current state vector.

In at least one embodiment, determining the second set of musical parameters of the second soundscape is based on a vectorial difference between a goal set of musical parameters and a current set of musical parameters, determined from a vectorial difference between the goal state vector and the second current state vector

In at least one embodiment, the method further comprises determining a current level of focus based on the current state vector, and the desired goal state of the user is a desired level of focus.

In at least one embodiment, the second set of musical parameters is determined by a machine learning model.

In at least one embodiment, the method further comprises collecting, in real time, the EEG data of the user to which the second portion of generative music is played and determining whether the level of focus is improved.

In at least one embodiment, the method further comprises determining a third set of music parameters of a third portion of the generative music which is more susceptible to force an improvement of a level of focus and transitioning the generative music automatedly generated into a third portion of generative music based on the third set of music parameters, and playing the third portion of generative music to the user.

In at least one embodiment, the server is coupled to another sensor configured to measure environmental data, and the method further comprises receiving environmental data from the electronic device and a context-relevant interaction data indicative of the user interaction with the electronic device and adjusting the second current state vector based on the received environmental data and the context-relevant interaction data.

In accordance with another aspect, there is provided a system for generating music for an electronic device, the system comprising: an audio stems database comprising a first plurality of audio stems and a second plurality of audio stems; and a speaker and a biosensor located in the electronic device, the sensor being configured to measure an electroencephalographic (EEG) data of the user, and the speaker being configured to receive and play a generative music. The server comprises a processor located on the server and the processor is configured to: based on comparing of a first current state vector having a first set of musical parameters with stem label vectors of the audio stems, retrieve a first plurality of audio stems from the audio stem database and generate a first portion of generative music by combining the first plurality of audio stems into a plurality of simultaneously played layers, the first portion of generative music having the first set of musical parameters; receive, from the biosensor, the EEG data while the first portion of generative music is played by the speakers to the user; determine, by analyzing the EEG data, a second current state vector that characterizes the second current state of the user; based on the determined second current state vector, determine whether a current state should be modified to achieve a desired goal state of the user by determining an error state vector; in response to determining that the current state should be modified, determine a second set of music parameters, for achieving the desired goal state of the user; based on the second set of music parameters, retrieve a second plurality of audio stems from the audio stem database, and combine the second plurality of audio stems to generate a second portion of generative music characterized by the second set of music parameters; and transmit the second portion of generative music to the speaker and collect, in real time, a second set of EEG data of the user measured while the second portion of generative music is being played to the user by the speakers.

In at least one embodiment, processor is coupled to an audio effects database comprising a first plurality of audio effects, and wherein the processor is configured to generate the first portion of generative music characterized by the first set of musical parameters further comprising combining the first plurality of audio stems with the first plurality of audio effects into a plurality of simultaneously played layers.

In at least one embodiment, the processor is configured to determine the first set of musical parameters of the first soundscape based on the first current state vector.

In at least one embodiment, determining the second set of musical parameters of the second soundscape is based on a vectorial difference between a goal set of musical parameters and a current set of musical parameters, determined from another vectorial difference between the goal state vector and the second current state vector.

In at least one embodiment, the process is further configured to determine a current level of focus based on the first current state vector, and the desired goal state is a desired level of focus.

In at least one embodiment, the processor is configured to determine the second set of musical parameters by a machine learning model.

In at least one embodiment, the server is configured to: collect, in real time, the EEG data of the user to which the second portion of generative music is played and determine whether the current level of focus is improved.

In at least one embodiment, the processor is further configured to determine a third set of music parameters of a third portion of the generative music which is more susceptible to force an improvement of a level of focus, and transition the generative music automatedly generated into a third portion of generative music based on the third set of music parameters, and the system is further configured to play the third portion of generative music to the user generated based on the third set of music parameters.

In at least one embodiment, the system further comprises another sensor coupled to the server and configured to measure environmental data, and the processor is further configured to receive environmental data from the electronic device and a context-relevant interaction data indicative of the user interaction with the electronic device and adjusting the second current state vector based on the received environmental data and the context-relevant interaction data.

In accordance with another aspect, there is provided a method for generating music for an electronic device having a biosensor and a speaker and coupled to a processor, the method comprising: based on biosensor measurement data received from a biosensor, generating a first portion of generative music by combining a plurality of audio stems based on a determined first current state vector; measuring, by the biosensor, biosensor measurement data while the first portion of generative music is played by the speakers to the user; in response to determining that the current state should be modified to achieve a desired goal state of the user, determining a second set of music parameters for achieving the desired level of focus of the user; and generating, by the processor, and play, at the speaker, a second portion of generative music characterized by the second set of music parameters to change the current state of the user.

According to an embodiment, the biosensor measurement data comprise electroencephalographic (EEG) data.

In accordance with another aspect, there is provided a system for generating music for an electronic device having a biosensor and a speaker and coupled to a processor. The system is configured to: based on biosensor measurement data received from a biosensor, generate a first portion of generative music by combining a plurality of audio stems based on a determined first current state vector; measure, by the biosensor, biosensor measurement data while the first portion of generative music is played by the speakers to the user; in response to determining that the current state should be modified to achieve a desired goal state of the user, determine a second set of music parameters for achieving the desired goal state of the user; and generate, by the processor, and play, at the speaker, a second portion of generative music characterized by the second set of music parameters to change the current state of the user.

### BRIEF DESCRIPTION OF THE DRAWINGS

Further features and advantages of the present disclosure will become apparent from the following detailed description, taken in combination with the appended drawings, in which:

FIG. 1 is a front view illustrating headphones having EEG sensors, according to an embodiment;

FIG. 2 is a schematic diagram illustrating the use of headphones having sensors to provide feedback for concentration, according to an embodiment;

FIG. 3 is a schematic diagram illustrating feedback to the user, according to an embodiment;

FIG. 4 is a schematic diagram illustrating an architecture of headphones having a plurality of different sensors, according to an embodiment;

FIG. 5 is flowchart illustrating a method collecting data with EEG sensors and extracting meaningful information from the data, according to an embodiment;

FIG. 6 is a flowchart illustrating a method for generating music based on a current level of focus and acting on the level of focus with the generated music, according to an embodiment;

FIG. 7A is a schematic diagram illustrating user and system interactions, according to an embodiment;

FIG. 7B schematic diagram illustrating user and system interactions, according to at least one embodiment of the present disclosure;

FIG. 8A is a flowchart illustrating a functional blocks for generating music based on a current level of focus and acting on the level of focus with the generated music, according to an embodiment;

FIG. 8B schematically illustrates user's states, state vectors with reference to soundscapes and musical parameters, in accordance with at least one embodiment of the present disclosure;

FIG. 9 is a flowchart illustrating a method of correction of the control system in a forward model, according to an embodiment;

FIG. 10 is a flowchart illustrating an iterative process in method of correction of the control system in a forward model, according to an embodiment;

FIG. 11 is a flowchart of a plurality of corrections applied in the control system in a forward model, according to an embodiment;

FIG. 12 is a flowchart illustrating a generic method of control in an inverse model, according to an embodiment;

FIG. 13 is a flowchart illustrating the music engine within the control system, according to an embodiment;

FIG. 14 is a flowchart illustrating how feedback is applied or produced by either awareness or entrainment, according to an embodiment;

FIG. 15 schematically illustrates an audio stem database and a set of musical parameters, according to an embodiment;

FIG. 16 is flowchart illustrating a method for generating music for an electronic device, according to an embodiment; and

FIG. 17 is flowchart illustrating a method for generating music for an electronic device, according to another embodiment.

It will be noted that throughout the appended drawings, like features are identified by like reference numerals.

### DETAILED DESCRIPTION

The present method involves neuro-adaptive music, in that the music being generated in an automated manner adapts to neuronal data, such as an electroencephalogram (EEG) or similar data collected on an individual and relating to their cerebral state. In other words, it involves using data from sensors to infer neural states of the user, and using these inferences to programmatically control a modular music soundscape in order to influence the user's state.

It should be noted that the terms “neural state” and “state” of the user are used herein interchangeably. It should be understood that “soundscape” and a portion of generative music is used herein interchangeably.

In an embodiment, data from EEG sensors (which are electrodes of an appropriate size and shape and located at the right locations on the user's head) are inferring the user's level of focus, and their measurements (output from the sensors) are used to modulate an adaptive music piece in order to improve the user's focus, hence the biofeedback in reference specifically with the user's focus.

In other words, the user's focus is used both as an input for adapting the music: more specifically, the output from the EEG sensors is interpreted and used (either raw data or interpreted to be transformed into focus indicators) as the input to adapt the music being generated in real time, and the finality is to improve focus of the same user to whom the music being adaptively generated in real time is being played.

Real time implies that an event is measured as it happens, without any significant delay, or an action is taken when the decision is taken and in a very small period of time after having collected the data which made the decision possible. Real time therefore involves immediate collection, immediate processing and immediate decision, taking into account the necessary small period of time for transmitting and analyzing the information and for performing an action. In the present context, “within seconds”, less than a minute, can be considered to be a real-time process. Music changes can also take a number of seconds to be operable in order to have time to make a proper transition in musical parameters (sudden changes to musical parameters may appear unnatural and a buffer period can be programed in the method to avoid such transitions too rapidly; however, the decision to change musical parameters is still performed in real time and the triggering of the period of transition of musical parameters to change the mood is also a real-time process).

The method described herein therefore involves the use of biosensing metrics to modulate (adjust) music; a method for modulating the music, wherein the way the method for modulating the music is designed to be modulable by biosensing metrics; and the use of said music being modulated to “manipulate” or influence their state of focus (or more generally their state of mind). The music is generated and modulated in real-time and as a feedback loop between the biosensing metrics being collected and the music being generated, in view of a target mood to be achieved with said music being generated in feedback to the biosensing metrics being collected in relation to a current mood.

### Stems

According to the present method, compositions may be generated with a control of the music in very granular detail. The compositions are generated by using musical soundscapes that loop through several musical stems to play a continuous background-like sound.

Stems may be instrumental and non-instrumental stems. The instrumental stems include music note(s) played on one of musical instruments. Non-instrumental stems may include vocal interpretation of the note(s) and other sound stems which may include various natural sounds, such as the sound of wind (for example, wind in a forest), water (for example, water flowing, or wave sounds), bird chirping, etc. The stems may also include pieces of synthesized and/or electronic music. For example, a stem may be 30-60 seconds, or even as short as a single note and therefore in the order of magnitude of a tenth of a second.

Stems may be played several times in a row on repeat to create a musical experience. For example, a stem may be a ˜2-3 second piano melody, or a short second drum beat, or a repeating background base line. If played alone, these stems would be very boring, but when several stems are combined together (for example: a melody stem, a rhythm stem, a polyrhythm stem, and an accent stem all played together at the same time) such combination of stems sounds like a short musical track that can be looped several times.

Some stems may be longer (˜30-60 seconds, or even more) while other stems are very short (even as short as one note). When using the shorter stems, the system needs to more frequently decide which stem to plan next, whereas during longer stems the algorithm may let the music play without changing anything. While one longer stem is playing, it's possible to add/remove other stems to compliment the first stem—for example, changing the drum track while the melody remains unchanged.

Using these stems, and based on pre-defined rules, the system combines several layers of sound together to create a track (also referred to herein as a “composition”). By removing layers, the track becomes less complex, because less instruments are playing at once. More layers will create a more dynamic and complex track. Because each stem is very short, the system is capable to add/remove stems on the fly (either sequentially over time or in parallel over simultaneous layers), as the user is listening, based on a decision matrix. This allows for the dynamic generation of music (tracks) following the rules.

As each stem may be as simple as 1 note and as short as several seconds, a composition may be formed by many stems. In addition, stems may be organized in layers, each layer having a plurality of stems. The system as described herein may then combine the layers into compositions by having the layers played simultaneously. Each layer may comprise two or more audio stems, and at least one layer of the plurality of layers may comprise at least one audio effect described further below.

For example, one composition may include about one thousand stems.

According to an embodiment of the disclosure, in absence of any measurements from biosensors, the stems can recombine randomly, i.e., there is a random determination of how the stems can be provided in a sequential, chronological order, taking into account basic rules of sequence combination to ensure that the sequence is musically consistent, such that the music would continue playing forever if undisturbed. According to an embodiment of the disclosure, such random transition between stems over a chronological sequence can be triggered simply for the sake of exploring the effect of other stems on the mood to avoid being locked in a local optimum for which other options would be preferable.

### Musical Parameters

In accordance with at least one embodiment, each sound composition (also referred to herein as a “sound track”) is characterized by a set of track values of musical parameters, and the values of musical parameters of the composition may be adjusted to control the perception of the music.

Each stem is characterized by a set of values of stem musical parameters. Each stem is labeled with a set of labels that correspond to the set of values of stem musical parameters. Such set of values of stem musical parameters is also referred to herein as a “stem label vector” L.

A set of musical parameters may comprise, for example:
Intensity, Bass; Bassdrive; Brightness; Number of notes played;
Energy qualities such as: Rhythm; Reverb;
Complexity level such as Polyrhythms; Number of rhythm layers; Number of tonal layers;
Mastering level such as High equalization (EQ); Mid EQ; Low EQ.

Other musical parameters may include, but are not limited to: warmth, richness, dynamic level, stereo width, intensity, tempo, rhythmic clarity, depth, harmonic complexity, density, frequency, prominence, polyphase, melisma, tremolo, volume, spectral balance, note density, entrainment frequency, reverb, etc.

In the set of musical parameters, each parameter has distinct impact on a users' emotional valence, physiological arousal, cognitive attention, perceived mood, or any other neural state which is desired to be manipulated. The musical parameters are qualities of the music, distinct of a specific melody, which impact the perception of the music by the user. These musical parameters may be pre-defined through specific musical definitions, or through subjective interpretation of the musical concept (such as, for example, a musician's gut feel).

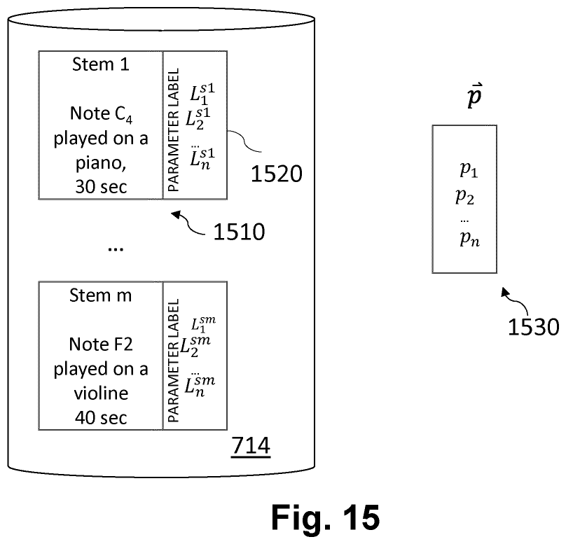

FIG. 15 schematically illustrates an audio stem database 714 having m stems, with each stem being labeled with a stem label vector L^{s1} . . . L^{sm}. In such stem label vector, each value corresponds to the value of one of the musical parameters of the stem. In FIG. 15, a set of musical parameters 1530 is also schematically illustrated.

In at least one embodiment, prior to influencing the user's mood, the audio stems database 714 is generated. Such database may include musical stems in pre-determined keys and genres. Genres may include, for example: piano, jazz, upbeat electronic, orchestral, etc.

In at least one embodiment, the system assigns a starting value to each one of the musical parameters. For example, a heavy drum rhythm stem may be assigned a high value of bass, bassdrive, warmth, intensity, and the same heavy drum rhythm stem may be assigned a low value of reverb, brightness, richness, and harmonic complexity. Starting values of the set of musical parameters may be randomly assigned at the start of the execution of the method, or assigned null values.

### Stem Versions and Effects

In addition to a database of stems labeled with the values of the musical parameters, the system also has a database of audio effects. For example, the audio effects may comprise filters, synthesizer effects, or other audio modifications that may be applied to a stem. Each one of these audio effects are labeled with values corresponding to the set of musical parameters.

Each stem may have several versions which has small differences. For example, one melody may have versions that are more intense than the other versions of the same melody. Such more intense version of the same melody may be generated, for example, by adding emphasis on certain notes. Alternatively, stem versions that are less complex than the other versions of the same stem may be generated by removing several notes.

The system as described herein may choose a stem version of the stem based on the desired style. For example, filters, synthesizer effects, and other musical modifications may be applied to specific stems to modify their sound in small ways, to achieve a specifical musical quality (increase brightness, warmth, intensity, complexity, etc.). Each new version of a given stem may differ from the other version of the same stem by a set of assigned values of the set of musical parameters, depending on the changes made to that stem to obtain such versions of the stem.

### Stem Combining Rules

A set of stem combining rules may be pre-defined by a musician prior to implementing the method as described herein, both with respect to the chronologica sequential combination of stems, and the combination of stems played simultaneously in parallel (layers). The stem combining rules define the ways that the stems may be combined to achieve a particular musicality of the composition. For example, in a given soundscape, a stem combining rule may request that the stems have only two rhythm tracks played simultaneously at a given time. In another example, another stem combining rule may limit which melodies may be played together. Another stem combining rule may force the stems in one soundscape to play at the same tempo. These initial stem combining rules are enforced such that any combination of the rules will still result in pleasant music. This helps to avoid musical dissonance which may interfere with the goal of the soundscape.

### Moods

The compositions also include several predetermined combinations of musical parameters (i.e., all these parameters are fixed in a given combination), meant to evoke a particular mood. The moods (each corresponding to a predetermined combination of the musical parameters) include, without limitation: “Alert”, “Energetic”, “Relaxed”, “Creative”, “Steady”.

Through the use of the system over time, additional moods can be determined from user feedback. These musical parameters and moods are selected for effectiveness in evoking such moods, i.e., it was found that these musical parameters have been demonstrated to have the most significant effect on a user's psychological and emotional state while listening to influence the user's mood toward one of the predetermined moods.

Adaptive or generative music is used to change the feeling of a piece of music in real time, based on external inputs. The method described herein involves a novel way of modulating this music based on focus-related data (i.e., from associated sensors), with the explicit goal of improving focus (for example, while the user performs their work) while listening to the music being generated in real-time.

Although the description mentions mainly “focus” as the primary state to be measured and improved, this could also apply to any passive biofeedback while the user is performing a task, to optimize the user's mood for that task (better focus, lower stress, better motivation, lower fatigue, etc.), i.e., any passive state that can be measured and improved.

The feedback is in real-time, i.e., the adaptation of the music being generated is based on the data as it is collected, and the adaptation is performed in real-time, such that the generated music is immediately adapted while being played to the same user from which data are collected, to apply the feedback in real-time.

### Biosensing

Biosensing involves using data from one or more sensors to infer the state of a user. Commonly used sensors include, without limitation, heart rate (PPG), movement (accelerometer), emotion (facial recognition), EEG sensors, muscle activity (EMG), eye movement (EOG), galvanic skin response (GSR), skin temperature, blood oxygenation, or any other biological interface or human-computer interface.

The data from these sensors, either individually or combined, is used to infer physiological states of a user. In the embodiments described herein, EEG sensors are used to infer attention, cognitive workload, motivation, fatigue, stress and mind wandering levels or states from the user. These inferences use machine learning algorithms to predict the likelihood of the user being in a given state.

The method described herein involves connecting the inferred states from these sensors to adaptive music, with the explicit goal of improving the user's focus while they perform any activity.

According to an embodiment, the EEG sensors are in a headset. This has many advantages, in particular that the user will willingly wear the headset continuously during many hours, and EEG data can be collected meanwhile to perform the biofeedback. An example of a proper headset for collecting EEG data is detailed further below.

In addition, or instead of EEG data, the same biosensor or one or more additional biosensors may include sensors that measure heartbeat, temperature of the user, ambient light intensity.

### Neuro-Adaptive Music

The method described herein involves connecting using biosensing to infer physiological (and psychological) states of the user, and in turn using these states to modulate an adaptive music composition which is adapted in real-time from the collected data and also played in real-time to improve focus as it is being measured by the sensors, with the explicit intent of improving the user's focus while they work.

The improvements in focus occur through several different, and complimentary mechanisms, including Sustained attention training and Direct entrainment.

### Sustained Attention Training

Sustained attention training (often called “alertness training”) involves using visual or auditory feedback to make the user meta-aware of changes in their neural state, with the goal of strengthening user's ability to recognize and mitigate distractions. This occurs by strengthening user's brain's sustained attention mechanism.

Using the method described herein, the system generates neuro-adaptive music which can use the inferred neural states (i.e., raw EEG data interpreted into a given level of focus/mental state) to change the adaptive music every time that the user's state, as determined in real time, changes. In this way, each time the user hears the music change which was triggered in real time, the user recognizes that this implies that their (i.e. the user's) neural state has also changed. This auditory feedback strengthen the user's level of meta-awareness of their neural states, and allow the user to return their attention to their task (i.e., improve their level of focus) each time they become distracted, or fatigued, or their engagement drops.

The process of automating this feedback, and providing the feedback through the changing of musical parameters, considerably differs from the more generic generative music used in other applications, such as in video games. Unlike most sustained attention training mechanisms, the implementation of the method described herein, according to an embodiment, is meant to be passive (in other words, used in the background while the user focuses on their work) rather than active (such as, for example, focusing on the music explicitly, with the purpose of training the user's focus).

Furthermore, in at least one embodiment, rather than providing feedback to the user through a beep, or a change in volume, the feedback is provided by a change of the soundscape which is controlled by adjusting musical parameters, such as, for example intensity or other musical parameters. For example, the feedback may be provided by increasing the intensity of one or more of the stems of the soundscape.

Alternatively, the feedback to the user may be provided by changing the “mood” of the music being generated in real time. Such change of mood may be based on the raw EEG data interpreted into a level of focus, the change of mood being driven by the determination that the level of focus is deemed to be insufficient, the change of mood comprising an automated, real-time change in the musical parameters of the music being generated and played in real-time to the same user being monitored. In this way, the information (meta-awareness of the user's own state of mind and/or level of focus) is conveyed to the user in a pleasant manner by letting the user know their neural state has changed, without further distracting the user from their task. As referred to herein, the meta-awareness means a state of deliberate attention toward the contents of a user's conscious thought.

### Direct Entrainment

It was found that certain musical parameters have significant and repeatable impacts on a user's psychological and emotional state while listening. In at least one embodiment of the present disclosure, this information is used to create a musical experience that entrains the user into a deeper state of focus while the user listens to the soundscape. In the embodiments disclosed herein, the musical experience which is obtained by playing the soundscapes to the user, is automated. Such automation is provided by generating the portion of music which has pre-determined musical parameters, where each successive portion of music (i.e., chronological succession, where each portion follows a previous one in time) is driven by a set of musical parameters which correspond to a “goal state” to be achieved within that portion of music.

The “goal state” is determined as being one which is consistent with both the current level of focus, as determined by interpretation from raw EEG sensors, and by a desired next level of focus, which can be either the same level as presently measured if it is already determined as satisfactory, or can be an improved level of focus if it is determined that an improvement is possible or desirable.

When the biosensors detect that a user is in a particular current state, a new soundscape with adjusted musical parameters may be generated to induce changes to the user's state. Alternatively, to induce changes to the user's state the previous soundscape may be modified by adjusting one or several musical parameters.

In at least one embodiment, the adjustment needed for the musical parameters may be explicitly coded. For example, if the user's current fatigue is high (the user is tired), the system may increase the intensity of the music, since such change (modulation) reduces fatigue.

In at least one embodiment, the adjustment needed for the musical parameters may be implicitly discovered by the system. For example, when the user's fatigue is high (i.e., the user is tired) and therefore the user's level of focus is low, a soundscape may be modified by randomly modifying the set of musical parameters by the processor of the system. The user's level of focus (and/or fatigue) is then measured by receiving and analyzing the data from one or more sensors (such as, for example, EEG sensor). If the soundscape with the adjusted set of musical parameters has improved the level of focus of the user (i.e., as a result of the change of the musical parameters, the user has better focus), the system remembers and keeps this set of musical parameters and applies it to the subsequent generation of the soundscapes.

Random sequences or random layering (i.e., random sequential combination and/or parallel combination) can be tried to find if the current situation is only a local optimum and to try to see if other randomly chosen combination may outperform a current combination already determined to be good. In some embodiments, the system, when executing the method, randomly but preferably avoids sets of musical parameters which were tried earlier by the system and have been determined to be unsuccessful. For example, such musical parameters that are known to be unsuccessful may be labeled and/or stored in an additional “database of musical parameters to avoid” (not depicted in drawings).

According to an embodiment, a predictive model is built. The predictive model maps music changes to changes in quantifiers which can be used to represent in a quantitative manner (that is measurement-based) the current state and target state of the user. There are a few options to achieve this as a forward and/or inverse dynamic model, as described further below. The predictive model is used inside a control system to evaluate the effect of the music on the brain “before” sending the actual feedback.

The predictive model also has a correction mechanism which may correct predictions in view of new data collected in real time. In addition or instead of the correction mechanism, the predictive model may have a “learning” mechanism.

Via the learning mechanism, useful prompts and “random” prompts are sent to analyze the effect on the brain of these individual prompts, both on their own, and also in combination with the music playing in the background. It should be understood that “random” prompts may be made randomly to try new combinations of stems, the randomness being both sequential and/or in parallel, and includes restrictions to avoid having purely random combination, by taking into account that some basic combination rules are necessary to ensure musical consistency (rhythm, tonality, etc.) of what is being played.

The algorithms for adjustment of the music in order to improve focus may be generalized such that, for example, the same rules that describe correspondence between the musical parameters and stem combinations, and the induced mood evolution as measured through suitable biomeasurements (or biosensor measurement data), may be used for everyone. Alternatively, the rules may be individually tuned to the user's preferences. For example, a model that is specifically optimized for a single user's brain may be trained. The rules may also be applied to a single listening session for a user, such that the music being generated is absolutely personalized, both for a single person, and for a single listening session, in view of the original mood as described by collected measurements from biosensor (typically unique to a person and at a particular time such as at the beginning of a listening session or during the listening session), and the target mood which can be predefined by the user with a label selection from a user graphical interface.

In at least one embodiment, when the user's current state and the desired (goal) state are known, and the control system varies the musical parameters in order to transition the user from the initial current mood (can be user-selected or can be inferred from actual measurements from biosensors) to the desired mood (or target mood, typically user-selected as mentioned above).

The closed-loop feedback mechanism may be either explicit or implicit, as described above. By providing the closed-loop feedback from the sensor (such as EEG sensor) to the processor, the system further applies the “error-minimization” routine in order to force the user to transition between a current state and a goal state. The error-minimization routine is described in greater detail further below.

### Music Personalization

Musical preferences are varied and are often unique to an individual. These preferences directly impact the desirability of the music, and therefore directly impact the ability for a musical soundscape to create the ideal environment for a user to focus on their work.

The measurements collected by the biosensors may be used, over time, to train a model to recognize the user's musical preferences over a significant period of time of use of the application which implements the method described herein. Based on such training, the model may tune a given soundscape to the user's preferences, with the goal of creating the optimal environment for the user to better represent their preferences.

In at least one embodiment, the soundscape may be generated based on the user's musical preference (e.g., favorite song), by modifying the user's preferred sound tracks and adding stems or removing portions of the track, or applying sound effects to the specific stems or the whole soundscape or the whole track to create new mixes that achieve the desired state. In another embodiment, the user's music preference is exclusively used, and the system selects the user's preferred tracks that can help the user to achieve the desired goal state.

The biosensors may be used to train a model to recognize the user's parameters which are the most effective for the user to focus on their work. The effectiveness is a goal which is different from the respect of user preferences. In such embodiment, the biosensing measurements may be used to personalize the musical soundscape to the user.

In such embodiment, the user listens to several musical soundscapes while they work, and the system observes the effect of the musical parameters on the user's focus by collecting EEG data during the trial period for that specific user. The system then analyzes the received EEG data to infer a set of musical parameters which correlates with the user's measured mood for that user within that trial period of listening time. The system then associates the affinity of a user with a particular musical (or soundscape) mood or style with respect to the improvement or sustainment of a level of focus when exposed to a music/soundscape of that mood or style (as determined by the corresponding set of musical parameters).

The songs that the user liked may also be analyzed to “uncover” the user's preference using their favorite playlist or have them select their favorite songs. The frequency distributions can then be analyzed, and the mood/style of the songs, tempo, and other features can be considered in the analysis to build a preference profile for that user.

In at least one embodiment, linearized models are built that map individual changes in the music to changes in the brain. The system then uses the linearized models to determine which music (or which changes in the music) are the most effective to trigger the desired changes in the user's brain and therefore to trigger the desired changes in the EEG data received by the processor.

In at least one embodiment of the control system, the described models do not need to be perfect to reach the destination. For example, such models may be simply “good enough” or, in other terms, the result of the implementation of the model may be within the pre-determined error margin. As a result of implementing such models, even though the music effect on the brain is highly non-linear, methods using simple individual linearized models permit changing the music, and results of implementation of these linearized models may be combined together. For example, such results of implementation of the linearized models may be a modified set of musical parameters. The result of the combination of the results obtained with the linearized models may be analyzed to estimate the final “effect” on the brain before applying the changes to the subsequent soundscapes.

Due to the feedback loop described herein, changes are made to the musical parameters of the soundscapes in real time while the user listens to the soundscapes and at the same time tries to focus. For example, such changes to the musical parameters may be made as an automated experimental trial, in order to learn what effect the changes in soundscapes (music) has on the user's focus. Such automated experimental trials may be implemented within a single work session, or during many work sessions.

In at least one embodiment, the focus level of the user may be determined passively by measuring the biosensor data (such as EEG data), receiving it by the processor and analyzing the biosensor data (in other terms, monitoring the focus of the user) while the user listens to any type of music, without any change. In such passive monitoring, the system may learn and correlate the user's focus level with the inherent musical variations that naturally occur in songs. Based on the qualities of the song played and such passive analysis, the system may determine such correlation without having to apply any changes (modify) at all to the music being played.

A reinforcement machine learning model may be used to learn what musical parameters are preferable to apply to the soundscapes for the user to be able to focus. In addition to the biosensing metrics, the user's direct feedback (for example, such direct feedback may be collected in response to a prompt to fill in a survey through a graphical user interface such as on a computing device which implements the method) may be collected and used to optimize choice of musical parameters.

For example, a clustering analysis may be applied to the data collected from several users in order to identify categories of users that respond similarly to changes in musical parameters, and create population-wide personalization. A machine learning model may be built and trained based on the results of such clustering analysis and may categorize a new user in the existing categories. The machine learning models may identify new musical parameters, and may help to identify design of new musical compositions optimized in real time for improving focus in view of measured data collected in real time indicative of such focus.

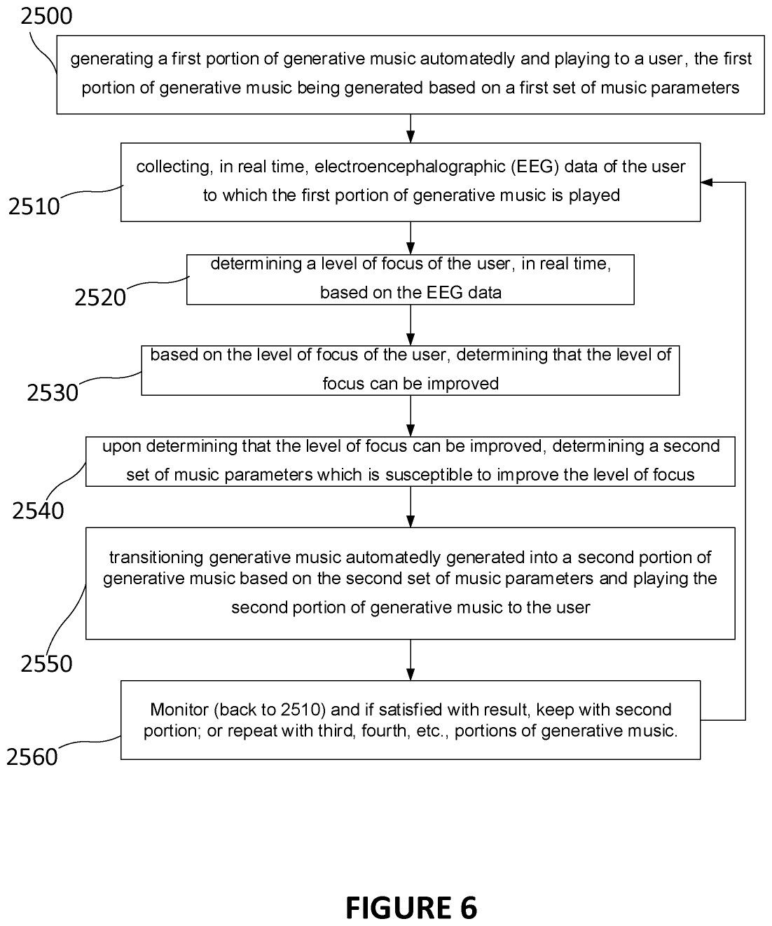

According to an embodiment, and referring to FIG. 6, there is shown a method for generating music based on a current level of focus and acting on the level of focus with the generated music, according to an embodiment.

Step 2500—generating a first portion of generative music automatedly and playing to a user, the first portion of generative music being generated based on a first set of musical parameters;

Step 2510—collecting, in real time, electroencephalographic (EEG) data of the user to which the first portion of generative music is played;

Step 2520—determining a level of focus of the user, in real time, based on the EEG data;

Step 2530—based on the level of focus of the user, determining that the level of focus can be improved;

Step 2540—upon determining that the level of focus can be improved, determining a second set of musical parameters which is susceptible to improve the level of focus;

Step 2550—transitioning generative music automatedly generated into a second portion of generative music based on the second set of musical parameters and playing the second portion of generative music to the user.

Step 2560—monitoring is performed (as in step 2510) and if the algorithm is satisfied with the result of the level of focus/mental state, the second portion is kept being generated with the corresponding set of musical parameters; otherwise, the changes are still triggered with third, fourth, etc., portions of generative music.

### System Overview

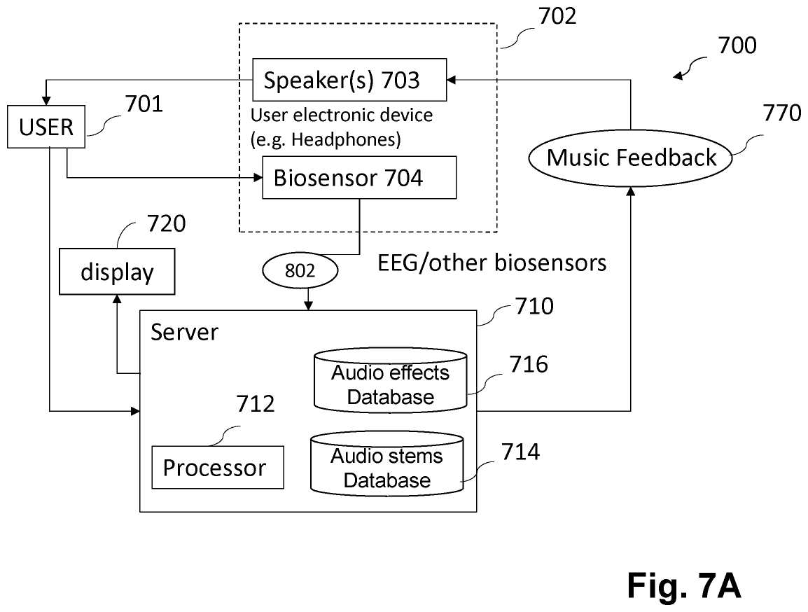

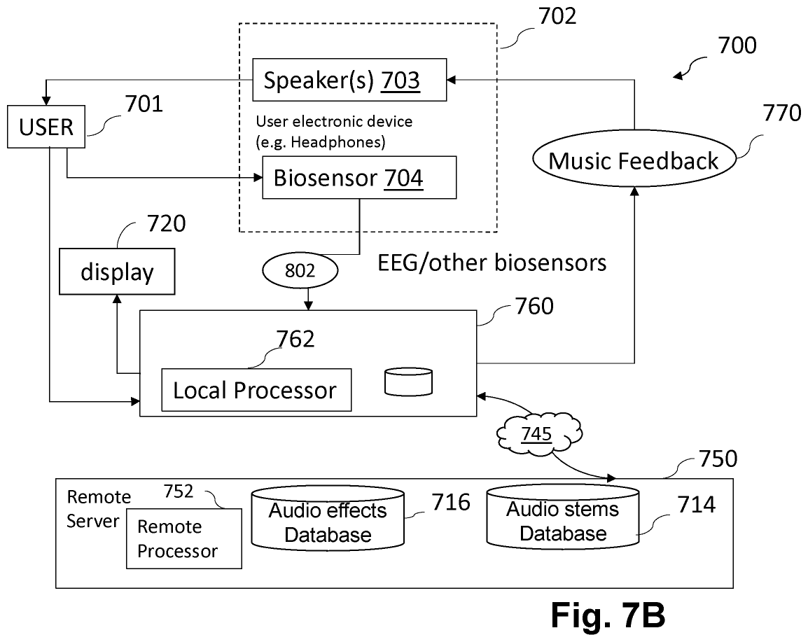

Now referring to FIG. 7, there is shown a system 700, in accordance with at least one embodiment of the present disclosure. The system measures and uses data descriptive of a user's biological state and the user's context-relevant interaction data to generate musical stimuli and influence the state of the user 701 to reach a pre-determined goal state.

The system 700 comprises an electronic device 702 of the user 701 which has at least one speaker 703 and a biosensing device 704. For example, and without limitation, the electronic device 702 may be implemented as headphones 100 described further below. The system 700 further comprises a server 710.

The server 710 has a processor 712 and databases such as an audio stem database 714 and an audio effects database 716. The server 710 may be also connected to a display 720 configured to display various prompt (requests) to the user 701.

The server 710 may be implemented as a portable or a stationary electronic device such as, for example and without limitation, a computer, a phone, a tablet, etc.

In some embodiments, some elements of the server 710 may be provided in various locations while those elements of the server 710 may communicate to each other via the internet or any other wired or wireless connection. For example, some steps of the method described herein may be implemented by a computer while the other steps may be implements by a remote server located on a cloud.

FIG. 7B illustrates such an embodiment of the system, where the databases are located on a remote server 750 connected to the computer 760 via a network 745. In such an embodiment, the computer 760 interacts with the user electronic device 702, and the steps of the method are partially performed on the computer 760 and partially on the remote server 750. For example, audio effects database 716 and audio stems database 714 may be located on the remote server 750. In some embodiments, the remote server 750 may have a remote processor 752 that may execute some steps of the method described herein. The computer 760 may have a local processor 762. In some embodiments, the local processor 762 may mostly enable collection of various data from the biosensor 704 and other sensors and communication between the electronic device 702, display 720 and the remote server 750.

The processors 712, 752, 762 described herein are configured to execute the instructions of the method described herein. The servers comprise hardware and/or software and/or firmware (or a combination thereof) to execute one or more applications.

The functions of the various elements shown in the figures may be provided through the use of dedicated hardware as well as hardware capable of executing software in association with appropriate software. When provided by a processor, the functions may be provided by a single dedicated processor, by a single shared processor, or by a plurality of individual processors, some of which may be shared. The illustrations provided herein represent various processes which may be substantially represented in computer-readable media and so executed by a computer or processor, whether or not such computer or processor is explicitly shown.

### Hardware and Application Installed Thereon

According to an embodiment, the embodiments of the method described herein are implemented on an application which is installed and run on a server 710 (or on two servers 750, 760 with reference to FIG. 7B). It should be noted that the server 710 (or server 760) may be implemented as a portable electronic device, such as a smartphone or a tablet, although any other computer (laptop, desktop, wearable device, etc.) can be suitable. The application can be downloaded from an online store and installed locally on the server 710, where the instructions which implement the method at run time are locally stored on a local memory.

The server 710 also comprises a processor 712 operable with the memory to execute the instructions. The server 710 should also include communication ports for receiving the collected data such as the EEG data and for transmitting the music being generated to the user electronic device 702, such as the headphones 100 as described above, and a speaker 703.

As mentioned above, in a preferred embodiment, it is the same user electronic device 702 which comprises the speaker 703 and the EEG sensors and/or other biosensors 704. For example, the speaker 703 may be embodied in the headphones with EEG sensors/biosensors of proper shape, format and positioning for collecting good-quality data.

Either a jack connector or a Bluetooth™ connection are suitable for the transmission of music generated on the spot by the server 710 and reception of EEG data from the biosensor 704 at the server 710, which contributes to the imminent automated changes made to the automatically generated music. Both the server 710 and the user electronic device 702 for collecting data and playing music are essential for carrying on the method. It is therefore possible that, in other embodiments, the speaker and the biosensors are separate (i.e., not part of the same device such as headphones). The speaker 703 could therefore be a self-standing type of speaker while the biosensors 704 could be any wearable device (watch, bracelet, headband, strap, sensors within clothing/hat/headgear, etc.)

The system 700 also comprises a feedback mechanism implemented by having the processor adapt the music being generated in real time in view of the data collected also in real time by the biosensors 704 to ensure that the initial music being generated is adapted to a current or initial mood of the user (as inferred from measurements and/or as indicated by the user), and also to ensure that the music being generated adapts and evolves over time (by sequential and/or parallel layering of stems) to have the user's mood, as inferred from real-time measurements, evolve toward a target mood as defined by specific biosensor data expected to correspond to this target mood. Music being generated not contributing to reaching this goal or target over time can be changed to adapt to the measurements until a proper stem combination is found which contributes to the chronological evolution of the current mental state measuring as being evolving toward parameters indicative of the target mood.

The user 701 wears the biosensor 704 while performing an activity on the computer. The biosensor 704 measures biosignals, such as EEG, and transmits the biosensor data 802 (such as, for example, EEG data) to the server 710. When the user 701 also works on the server 710 (which may be implemented, as discussed above, as a computer), the server 710 also collects context-relevant interaction data and environmental data. The server 710 processes the received data to extract characteristics of the user's current neural state. Based on the determined characteristics of the current state, the system provides two methods of feedback, entrainment, and discrete prompts.

The biosensor data 802 received by the server 710 may include heartbeat, temperature of the user or ambient temperature, ambient light intensity. The server 710 may have an additional sensor or a software determining computer activity or user activity on the server 710 (for example, the computer which receives, via Bluetooth, the collected biosensing data from the headphone 100).

According to an embodiment, the context-relevant interaction data can include interaction data of the user on the computer or user electronic device 702 on which the application runs. It can monitor user activity such as the use of specific applications, e.g., the application running the method can monitor that text editors, office applications, and other software work tools are being used on the same user electronic device 702; or that leisure-related applications such as movie streaming, video games, social media and the like are being used; or that the user electronic device 702 is not being used. This can be used as an additional input to determine the current state, or as an input to re-prompt the user about their target mood if there is an inconsistency between the set target and the current use of the computer resources, or as a prompt to bring the user back to the right track. For example, is the set target is deep focus and the user has started video games, then the target may need to be redefined, or if the user is using a social network, he may need to be prompted to go back to work. Monitoring user activity by having the application collecting current use of computer resources therefore contributes in determining if the user's mood is evolving appropriately, in addition to the collected biosensor data.

### Selecting Current Mood

When the generative music starts playing to the user, or prior to hearing any music, the user may select their current mood from a list, as well as their desired (target) mood for the session. These moods include, but are not limited to: happy, sad, excited, anxious, calm, intense, frustrated, eager, alert, etc. The user's selection of the “current mood” is used to collect information about the neural signature of this mood. In other words, it helps to map the combination of the neural states and the state vector to the current mood selected by the user. The user's desired (goal) mood sets a target for the specific combination of neural states that the system can achieve during the execution of the method as described herein using the music.

Each mood is mapped to an initial combination of musical parameters, which is used by the system to start the generative music. In other words, if the user selects a current mood of “happy”, the system retrieves the combination of musical parameters that corresponds to the mood “happy” to use such combination during the session. This ensures that the music which is used to start the session reflects the user's current neural state, to provide a baseline for the neural state measurements, and to ensure that the music does not feel cognitively dissonant which would cause discomfort and skew the results.

### Processing of Data

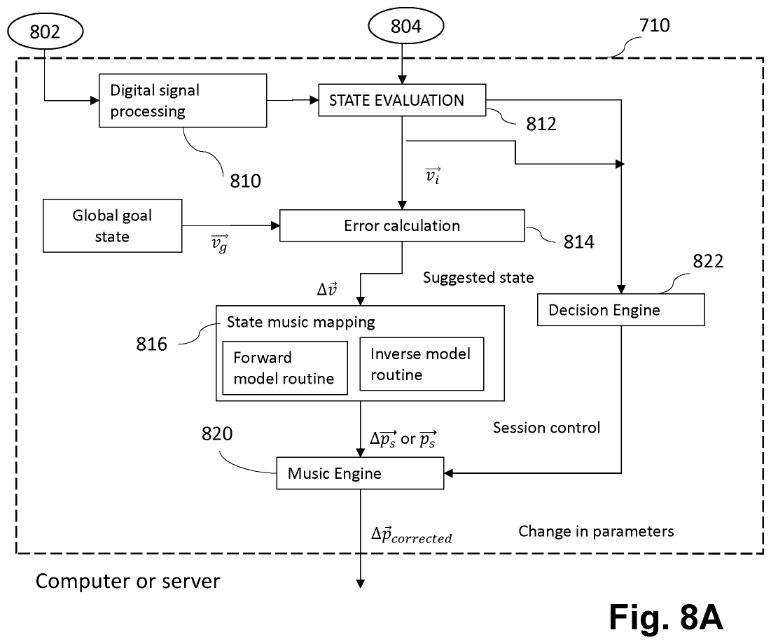

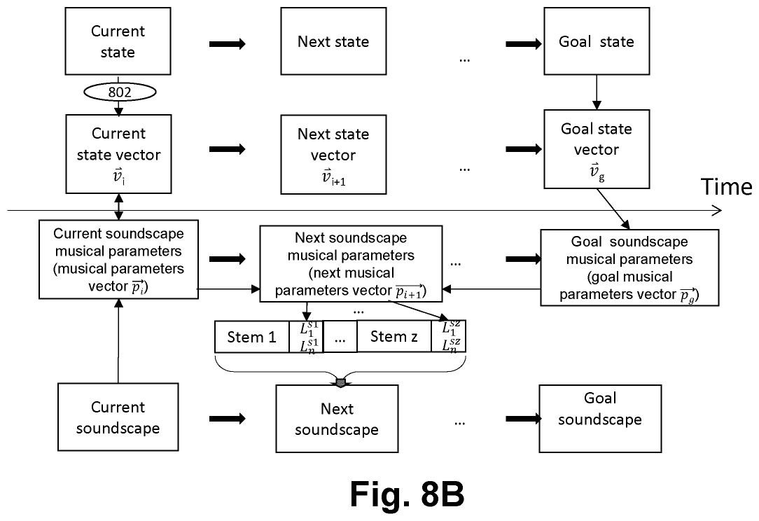

Referring now to FIG. 8, where a flowchart illustrates functional blocks for generating music based on a current level of focus and acting on the level of focus with the generated music, according to an embodiment. It should be understood that the functional blocks as described herein (such as digital signal processing block 810, state evaluation block 812 and other blocks described herein) may be implemented by software or a combination of hardware of the server 710 (or servers 750, 760) and software.

After receiving the biosensor data 802, the server 710 manipulates the data by the digital signal processing block 810 to generate a clean signal which is cleaned from various noises from various noise sources.

After cleaning the signal, the system 700 performs a state evaluation at the state evaluation block 812 to determine a state vector from the biosensor data 802 (biosignals). The state vector provides information about mood, focus, etc. as described below.

When describing the functional blocks of FIG. 8A, reference will be also made to FIG. 8B, where the user's states, state vectors are schematically illustrated with reference to soundscapes and musical parameters, in accordance with at least one embodiment of the present disclosure.

### State Evaluation

The state evaluation block 812 establishes a snapshot of the user's current state in the form of a n-dimensional state vector. The state vector at time i (also referred to herein as a current state vector \vec{v_{ι}}) may be expressed as follows:

$$\vec{{v}_{ι}} = \{ \begin{matrix}{v}_{1} \\ {v}_{2} \\ ⋯ \\ {v}_{n}\end{matrix} \} . \qquad ( 1 )$$

Each state parameter (v₁ . . . vₙ) in the state vector v_{ι} represents one aspect of the user's current state or context-relevant interaction data. An analogy may be made between the states of the system as described herein and a physical (mechanical) system. The state parameters of the system and method as described herein may be: attention, cognitive workload, alertness, motivation, fatigue, mind wandering, blink rate, heart rate, and so on. One may understand the state parameters as coordinates in a coordinate system of the states. Each value of a set of the state parameters of one user provides a “position” of the user's state in a coordinate system of the states, similar to x, y, z positions in a mechanical coordinate system. With this similarity in mind, one may apply control system methods and classical mechanics to control this state.

The state evaluation block 812 determines the user's neural state along the axes described above: motivation, fatigue, mind wandering, focus, cognitive workload, cognitive engagement, stress, etc. The state evaluation block 812 may also receive and use, in determination of the user's state, other data 804 about the environment, such as, for example: accelerometer, heart rate, galvanic skin response, environmental noise, auditory environment, light environment, computer interaction, mouse movement, keyboard strokes, current app usage, device connectivity, etc.

The state parameters may be determined based on the information received in various ways, such as:
EEG quantifiers: EEG data may be quantified by neural correlate parameters determined in offline controlled setting such as experiments.
Artificial intelligence models: State parameters may be extracted using machine learning models that are trained on user population data;
Derivatives: A rate of change of state vectors may be determined based on the past state and current state.
Subjective feedback: Information received directly from the user as an input in response to a prompt displayed to the user to select their perceived state;
External Data Source: Additional biosensing data may be received based on the registered user's activity or sleep trackers;
Individual Calibration: User-specific tuning parameters;
Environment: Time of the day and external context;
Computer Interactions: App switching, Current App used, etc.

Each state vector corresponds to a particular state, such as mood and level of focus. For example, the system may have pre-determined mapping grid for mapping scalars of the state vector to various moods and levels of focus.

Once the current state vector is determined, the system implements the method described herein to influence the change in the user's state and therefore to change the state vector based on music feedback.

### Global Goal Definition and Error Calculation

The purpose of the method described herein is to influence the user towards a defined desired (or target) goal state, which is considered to be optimal for the purpose the user has intended, starting from an initial state and continuously going through current states over time which are inferred measurements from the biosensors and monitored over time to determine if they evolve toward the target state or not using a linearized model to represent the states.

Regarding the target state, and also the initial state, for example, in response to a request (prompt) provided to the user, the user may select, for example, “focused deep work”.

The desired goal state may be determined by polling the user directly (by displaying a prompt to select the desired goal state from a list of states), or experimentally from previous sessions, using measurements from the biosensors. These goal states may then be modified by the following adjustment parameters:
Task: The goal state may be determined or adjusted depending on the type of task the user is trying to accomplish as determined based on the interactions with the server 710;
Desired Mood: The goal state may be determined or adjusted based on the user's desired mood, productivity, or motivation;
User profile: The goal state may be determined or adjusted based on user profile, received by prompting the user to fill in the survey;
Current time: The goal state may be determined or adjusted based on the time of day, or day of the week.

After determining the goal state, an error vector between the goal state and the current state is determined at an error calculation block 814. In at least one embodiment, the error vector Δ\vec{v} may be determined as:

$$Δ \vec{v} = \vec{{v}_{g}} - \vec{{v}_{ι}} \{ \begin{matrix}keep as vector \\ flatten as scalar\end{matrix} , \qquad ( 2 )$$

where \vec{v_{g}} is the goal state vector and \vec{v_{ι}} is the current state vector. In other words, to determine the error vector, the difference between the goal state vector and the current state vector (as determined from biosensor measurements) is calculated. The error vector is then flattened as a scalar (e.g., the module is calculated, or other similar scalar measures from a vector).

This error vector (or its module or other forms of scalar numbers) is then minimized by generating soundscapes as described herein and playing them to the user to stimulate changes in the user's state. Measuring whether or not the current state, as determined from biosensor measurements, evolves toward the target vector or not will determine if the musical parameters being selected and stem combinations will be continued, or changed to other variations deemed more appropriate to induce the required status change.

### State-Music Mapping

The system and method as described herein uses the effect of music on the brain. A state-music mapping block 816, uses the error vector Δ\vec{v} determined earlier to determine a set of musical parameters for the next soundscape to be played to the user in the next attempt to achieve the goal state of the user.

The method described herein may use one of two following routines to determine the stimulus required.

A forward model routine may be described as follows:

Δ\vec{v}=ƒΔ\vec{p},
where ƒ is a transfer function between the musical parameters \vec{v} and the state \vec{v}.

An inverse model sub-routine may be described as follows:

Δ\vec{p}=GΔ\vec{v},
where G is a transfer function between the state vector \vec{v} and the musical parameters vector \vec{p}.

The forward and the inverse model routines attempt to predict the relationship between the music (soundscapes) and the brain. In other terms, the forward and the inverse model routines may determine the relationship between the user's state represented as a state vector and the musical parameters vector of the soundscapes. The forward and the inverse model routines form a music feedback control system, as it determines how to generate the music in order to create the desired impact in the user.

Implementation of these model routines use a combination of experimentation, data obtained from literature review, big-data analysis, and other techniques that will be described below. The implementation of these model routines may also be modified by the adjustment parameters described above, including user feedback, the task, the desired mood, the user profile, and the current time.

### Forward Model:

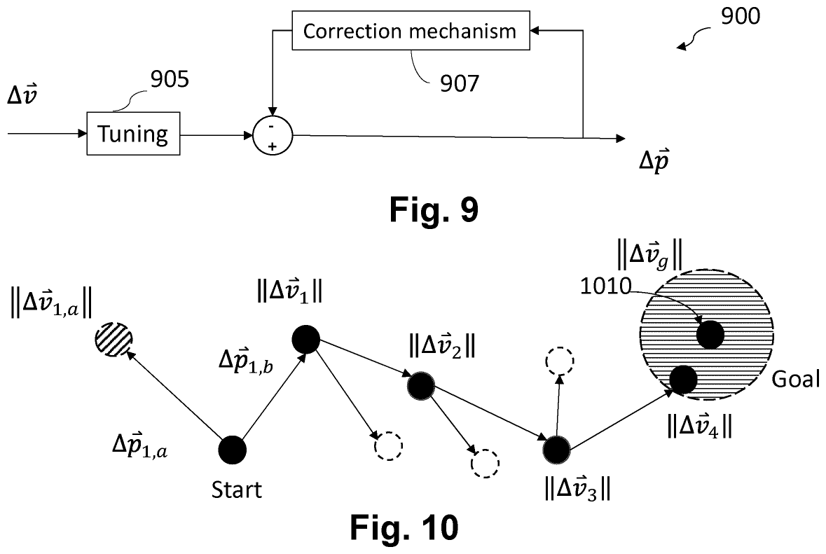

Now referring to FIG. 9, the forward model allows to predict the neural effect on the user that can occur due to sending a specific musical stimulus. In other words: the forward model involves identifying which changes in neural state will result from a given change in music.

Implementing the forward model involves training such a model to predict the impact of each of the musical parameters on each of the neural states measured. This model may be trained using a combination of experimental data and theoretical understanding.

The forward model is built using a weighted combination of the following data:

Data obtained from a literature review: Based on a literature review, the neural effects of certain musical parameters are known. The relationships between the musical parameters and the state characteristics form the basis for creating a model that determines the neural effects of certain musical parameters.

Experimental data: running tests with the users, wherein the effects of changes in musical parameters are monitored under controlled conditions. This may involve having the users complete specific tasks while the music is controlled, and measure the resulting changes in neural state. It may involve changing musical parameters one at a time (in order to isolate the effects of each parameter) or several at a time (in order to isolate the joint effects of musical parameters).

Real-world data: as users listen to the music, and as musical parameters are frequently changed, the effects of these changes can be monitored to update the model. In this way, as many users may use the invention, multiple examples of changes in neural state due to changes in musical parameters may be recorded. As such, the implementation of the model is self-correcting: every time a musical parameter is changed, the neural effect of the change is monitored, and used as data to improve the accuracy of the future mapping between the musical parameters of the soundscapes and the measured neural states.

As such, the method which implements the forward model may robustly determine a predictable neural effect from a given change in musical parameters.

In order to implement this forward model, a forward model mapping database must be generated and used to map the musical parameters to the state of the user. Similarly, the same or similar database may be used to map a change in musical parameters to a change in neural state of the user. Since the mapping of musical parameters to neural states is highly non-linear, an inverse transformation is impossible. As a result, a two-part process having two routines is used to substitute the inverse transformation.

FIG. 9 depicts a flowchart 900 illustrating an implementation of correction of the control system using a forward model routine. First, a “tuning” sub-routine 905 applies a linear transformation on the error vector, in order to produce the best estimate of the desired change in musical parameters. To implement such transformation, the system maps each individual neural state to a linear combination of musical parameters.

Second, a correction mechanism sub-routine 907 implements a “correction mechanism” by applying numerical methods to converge to an optimal choice of musical parameters. This involves iteratively changing the musical parameters, and using the forward model to validate the expected neural outcome from each selection.

The iterations of adjusting the musical parameters may be continued until the expected effect of the musical parameters of the soundscape on the user's state and therefore on the measured biosensor data is sufficiently close to the required error vector Δ\vec{v_{g}} 1010, as shown in the iterative process of FIG. 10. These iterations are implemented by the system and are invisible to the user. There are no indications sent to the user during the implementation of this step—only refining of the parameters based on “expected” effect on the user. The real response of the user and the change in the state of the user may be determined after sending the music and measuring and recording the EEG response.

The system mentioned above can be generalized to leverage multiple simultaneous models, either linear or non-linear. Models may be built and implemented for each musical parameter and provide a scalable system and the addition of new parameters over time. This technique is used to combine models trained based on different sources (literature review, experimental data, real time data).

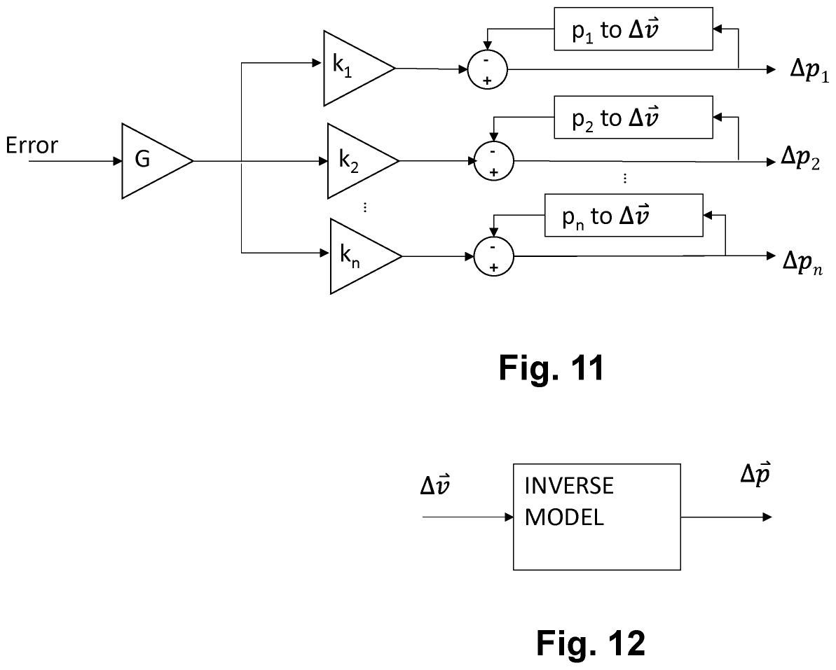

FIG. 11 is a flowchart of a plurality of corrections applied in the control system in a forward model. In this case, the “G” parameters, as shown in the schematic diagram of FIG. 11, is a global gain to control the magnitude/intensity of the response of all models, while each individual coefficient “k” (k₁, k₂, . . . kₙ) tunes the contribution of each individual model to compensate for non-linearities between the models and interactions not taken into account.

### Inverse Model:

In contrast to the forward model, an inverse model may also be used, as shown in FIG. 12. The inverse model involves determining a set of musical parameters that should be changed in order to yield a given set of neural states. In other words, the inverse model involves identifying what changes in music are necessary to achieve a desired change in neural state.

According to an embodiment of the disclosure, the method as described herein implements the inverse model through the use of moods: pre-determined neural states, for which the ideal musical parameters are known. These moods may be, for example: energetic, relaxed, creative, alert, etc.

The inverse model is trained as follows with the following data:

Data collected based on a literature review: As certain musical themes are known to induce certain moods, a moderately robust mapping of musical parameters to moods may be created from the data available based on the literature review.

Data collected experimentally: tests may be run with users, in which external feedback is used to induce a certain mood in the user. For example, the system may display to the user an invitation to perform a meditation exercise to induce relaxation. In another example, a brainstorm problem may be displayed to the user to induce creativity. Musical parameters may be thus changed while the user is in the specific state and/or mood (for example, creative or relaxed), in order to identify an ideal combination of musical parameters for inducing and sustaining the desired state and/or mood.

Real-world data: while one or more users listen to the music, the effects of the music on the users may be monitored over time. When the system determines that the user is in a given state and/or mood, the system may record the combination of musical parameters that led the user to this state. As more data is generated, the preferable combination of musical parameters necessary to induce a given mood may be determined.

Big data: with sufficient data, a mapping of musical parameters to neural states for all neural states (not only those defined by moods) may be learned and corresponding mapping database may be generated and then used by the system. This would simplify and add robustness to the implementation of the method. However, the implementation of the inverse model based on the big data needs the collection of substantial quantities of data.

The method as described herein may use the inverse model and forward model interchangeably, or in conjunction with one another.

In at least one embodiment, at the beginning of a session, the user is prompted to identify (for example, select from a list) the mood they desire to attain during the session, and the inverse model is used to determine preferable musical parameters for inducing such a mood. As the session continues and the user's neural state changes, and those changes are determined by the system using the measured biosensor data, the forward model is then used to determine what changes toned to be made to the music in order to maintain the ideal (preferable) neural state (the desired mood, or otherwise) for this session. As such, the user benefits from the precision of the inverse model, as well as the flexibility of the forward model.

### Decision Making—Control Interface

In at least one embodiment, the system has a control interface between the user and the computer application. The control interface may determine, based on user's EEG or other biosignals, triggers in order to control execution of the method. For example, in response to determining that the user state is beyond certain threshold (for example, if user's EEG data is beyond a pre-defined threshold), the system may be configured to terminate the execution of the method. In some embodiments, the system may perform customized actions in response to changes in the EEG data (for example Play/Pause of the music may be controlled based on the measured EEG data). Such a decision engine 822 is shown in FIG. 8A.

### Music Engine

Referring again to FIG. 8A and FIG. 8B, the output from the state music mapping block 816 is a determined musical parameters vector for the next soundscape \vec{p_{ι+1}}. This next musical parameters vector is transmitted to a music engine 820. The purpose of music engine 820 is to use the determined musical parameters vector and to generate music (the soundscape) according to these musical parameters in order to attain the goal state or to maintain the goal state. In other terms, due to the music engine 820, the feedback loop is implemented.

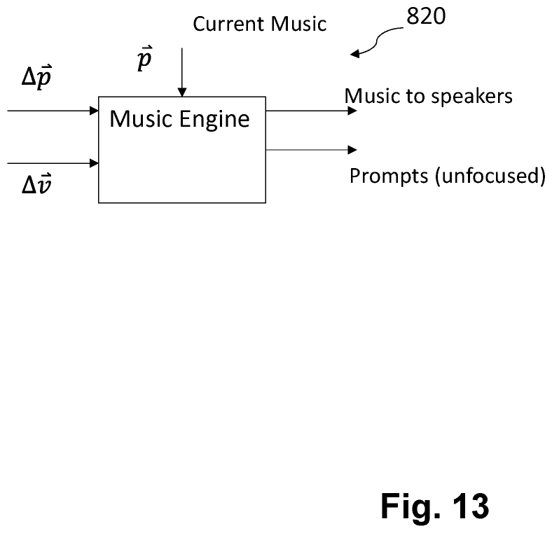

Now referring to FIG. 13, the music engine 820 generates soundscapes and manipulates them according to the data (musical parameters) received from the state music mapping block 816. This functional block may be implemented using adaptive generative music.

The music engine 820 may also implement rules and may reduce the number of parameters to only implement some parameters at a time. In various embodiments of the present disclosure, the music engine may:
Determine a sub-set of musical parameters that needs to be changed;
Generate musical structures and styles (such as, for example, rules);
Update the State-Music Mapping models (such as, for example, by reinforcement learning);
Generate soundscapes in real time;
During passive tracking, analyze the music playing and determine how it affects the user's states (for example, whether the mood changes or the level of focus improves or deteriorates);
Determine which musical parameters to implement at a given time;
Control transition timing and duration of application (length) of musical parameters;
Send prompts when the system determines that the user has reached a threshold state (for example, the user's focus is unacceptably low as determined based on pre-defined threshold parameters for the data received from biosensors);
Adjust a mood of a song (for example, by adding an additional layer to the track with audio effects, by changing a stem into a variation of that stem which corresponds to another mood, or by continuing a sequence at the end of a stem by another stem which corresponds to a mood);
Use existing songs to generate mixes designed for state improvement based on individual user preferences and their state;
Generate voice prompts or guided messages;
Integrate random/selected parameter changes for improving the predictive models;
Extract rules from existing songs to create additional moods and style;
Classify songs, tracks, soundscape portions and compositions (the latter being made of music portions such as stems, more complex tracks, including vocal songs, or soundscape portions) based on their effect on the brain and create specific playlists.

### Music Feedback

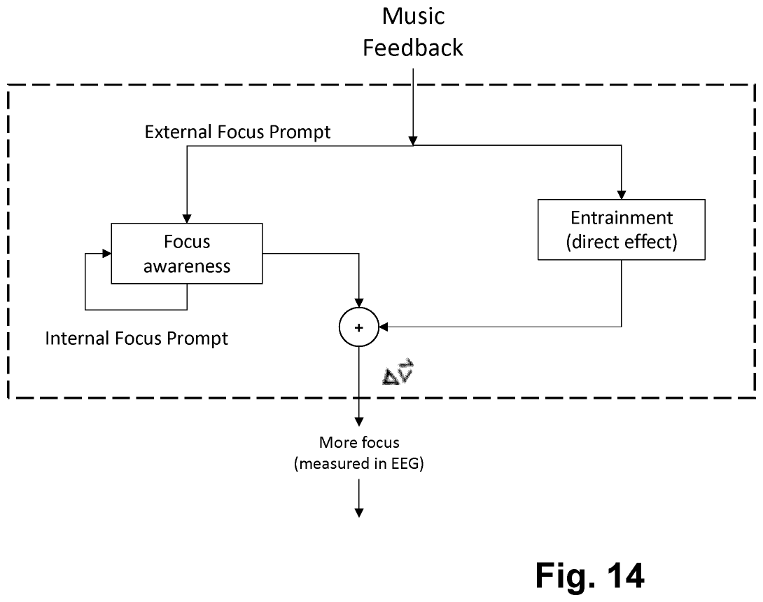

Referring now to FIG. 14, after the soundscape is generated and played through the speakers and thus stimulates the user, the state of the user may be improved by applying at least one of the two routines: entrainment routine and awareness routine, as shown in FIG. 14.

The entrainment routine is based on entrainment. Entrainment is the direct effect of the music on the brain, as predicted by the forward or inverse models discussed above. That is, the music directly causes a change in state in the brain.

The awareness routine is based on awareness mechanism. As the user receives feedback from the music notifying them of their current state (for example: distracted), they learn to recognize their own state and develop internal correcting mechanisms. Those mechanisms are always at work in parallel to entrainment, and are reinforced/trained by the external prompts. While the feedback is direct, both the awareness and the entrainment routines also take into account the subconscious changes in the music described above as “Sustained Attention training”.

To implement the awareness routine, the system displays a notification or plays an audio notification to the user based on the current state of the user determined using the biosensor data. The biosensor then measures the data and the biosensor data is then analyzed by the system to determine any change in the user's status.

The effect of implementation of these two routines—entrainment and awareness—is then recorded at the next step in time and a new state of the user is computed. A new set of musical parameters (musical parameter vector) is used to generate new, adjusted soundscapes and play them to the user.

The process repeats until the user or the system decides to terminate the musical experience. In other terms, the system may terminate the process internally, based on the pre-determined threshold parameters, or based on an external termination request received from the user.

### Closed Loop

In at least one embodiment, the system 700 adjusts the soundscapes by determining a goal set of the musical parameters and combining stems into a soundscape that is characterized by the determined next musical parameter vector (FIG. 8B) which is closer to the goal musical parameter vector. Preferably, the next musical parameter vector is within an error margin to the goal set of musical parameters (i.e., goal musical parameter vector). Based on the goal set of the musical parameters, the system 700 determines which stems should be played in the next soundscape, and which filters should be applied to these stems in order to generate a soundscape with the determined next musical parameters vector. In this way, the music (i.e. the generated soundscapes) transitions from a combination of stems that map to the user's current mood, into a combination of stems that is novel and different.

The new combination of musical stems in the next soundscape, which is characterized by the next musical parameters (FIG. 8B), will have an impact on the user's neural state.

After playing the next soundscape to the user via the speakers, the user's modified neural state is determined (in other terms, characterized by the system) based on measured EEG data (and/or other biosensing data), which is classified into neural states as described above. By monitoring the change in neural state of the user, the system determines whether the adjustments/changes in the soundscape based on the adjusted musical parameters were successful in leading the user to the desired goal state, or unsuccessful and leads the user to a different neural state.

The system 700 thus learns and adjusts mapping of the musical parameters, stems, and musical effects to changes in the user's neural state. Such a closed loop music generation system creates musical soundscapes that are personalized to a user, and trained to induce the desired neural states.

In at least one embodiment, the system 700 may test the impact of certain musical parameters or stems on the user's neural state by randomly changing the desired musical parameters or by selecting random stems from the stem database and combining stems into soundscapes randomly. This helps the system 700 to uncover patterns in musical parameter combinations or stems that may be beneficial or impactful to the user.

The data collected for various users may be stored in a training database and compared to discover combinations of musical parameters or stems that are generally associated with certain moods or neural states. This data may be retrieved and used to determine which stems to use, or which tracks to employ in subsequent iterations.

The impact of each stem used in the soundscape on the user's neural state may be compared to the recorded impact of one or more other stems that have the same values of musical parameters (in other words, have equal or similar music parameter labels 1520). Based on this information, the system 700 may modify the musical parameter rating of the stems, to better understand which stems have similar impacts to others, and subsequently better label these stems with their respective musical parameters.

### Clustering

Clustering algorithms may also be used to group the stems into new categories of stems which result in similar impacts on the user's neural state, but which do not correspond yet to a pre-determined musical parameter category. Based on collected data, new musical parameter categories may be created. In other words, such analysis may add one or more new musical parameters to the set of musical parameters. Such analysis may be applied to all users, or specifically to a single user.

This method may be applied to several genres of music, such as, for example, piano, jazz, rock, ambient, electronic, upbeat, classical, orchestral, etc. These stylistic differences may be kept separate. For example, one soundscape may have stems of only one genre. The genre may be decided explicitly by the user and selected by the user in response to a prompt displayed on the screen, prior to the user listening to the music. Alternatively, one soundscape may have stems of different genres of music to add variety. Yet in another embodiment, two subsequent soundscapes may have different genres.

### User's Feedback

The system may receive user feedback data from the user at any time during the execution of the method. Such user feedback data may include the information the system, while executing the method, is achieving the desired mood in the user. Based on such user feedback data, the system may then add a training label to the learning algorithm. Based on the user feedback data, the system may determine whether to continue to apply similar adjustments to the musical parameters, or to change the approach.

For example, if the music is repeatedly not achieving the desired mood/state in the user, the system may determine that several parameters need to be changed drastically and that a whole new musical environment needs to be created. For example, based on the determined user's state and the difference between the current state and the previous state, the system may determine that the genre of the music of the soundscape needs to be completely changed. This helps to avoid achieving a local maxima, such that no small change in musical parameters can achieve the desired change in the mood or the state of the user.

The system may receive additional input from the user to improve personalization of the music (soundscape) to the user. For example, the system may prompt the user to select what kinds of songs typically help to achieve a desired mood. The system then may associate certain musical parameters with certain moods from the start, thus improving the method's implementation.

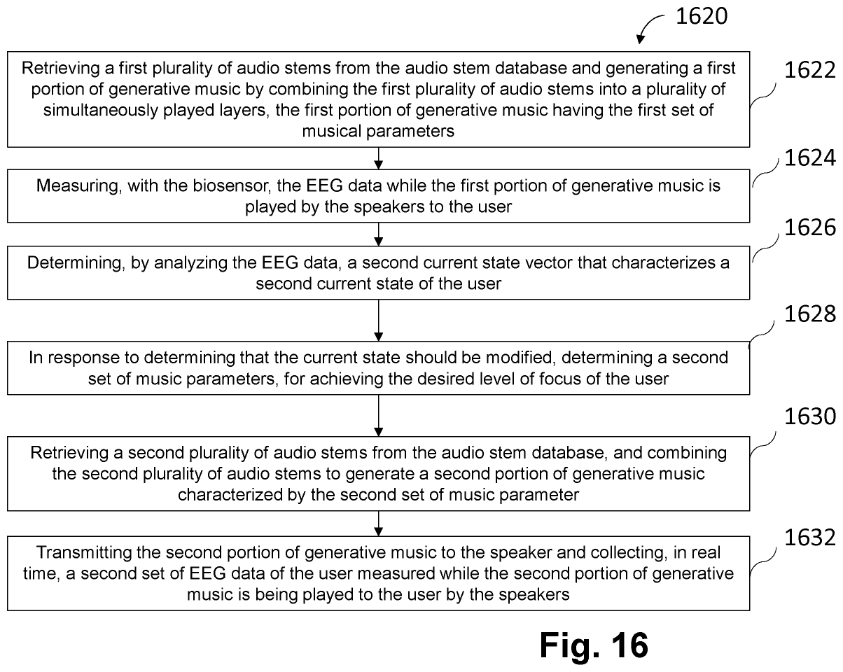

Referring now to FIG. 16, which depicts a flowchart illustrating a method 1620 for generating music for an electronic device, in accordance with at least one embodiment of the present disclosure. When describing FIG. 16, reference will also be made to the embodiments of system 700 depicted in FIG. 7A, 7B.

At step 1622, the system 700 retrieves a first plurality of audio stems from the audio stem database 716 and generates a first portion of generative music by combining the first plurality of audio stems into a plurality of simultaneously played layers. The first portion of generative music has the first set of musical parameters. Prior to retrieving the first plurality of audio stems, the system 700 determines a first current state vector having a first set of musical parameters. Based on a comparison of the first current state vector with the stem label vectors of the audio stems, the system 700 retrieves a first plurality of audio stems from the audio stem database and generates, by the processor 712, a first portion of generative music by combining the first plurality of audio stems into a plurality of simultaneously played layers, the first portion of generative music having the first set of musical parameters.

At step 1624 the biosensor 704 measures the biosensor data (such as the EEG data) while the first portion of generative music is played by the speakers to the user.

At step 1626, the processor 712, 752 determines, by analyzing the EEG data, a second current state vector that characterizes a second current state of the user.

The processor 712, 752 then, based on the determined second current state vector, determines whether a current state should be modified to achieve a desired goal state of the user by determining an error state vector. At step 1628, in response to determining that the current state should be modified, the processor 712, 752 determines a second set of music parameters, for achieving the desired level of focus of the user.

At step 1630, based on the second set of music parameters, the processor 712, 752 retrieves a second plurality of audio stems from the audio stem database, and combines the second plurality of audio stems to generate a second portion of generative music characterized by the second set of music parameters.

At step 1632, the system 700 transmits the second portion of generative music to the speaker depicted in FIGS. 7A, 7B as the music feedback 770, which is the music being generated in a way which is adapted to the measurements being made in real time, hence the feedback. At the same time, in real time, the system collects a second set of EEG data of the user measured by the biosensor 704 while the second portion of generative music is being played to the user by the speakers.

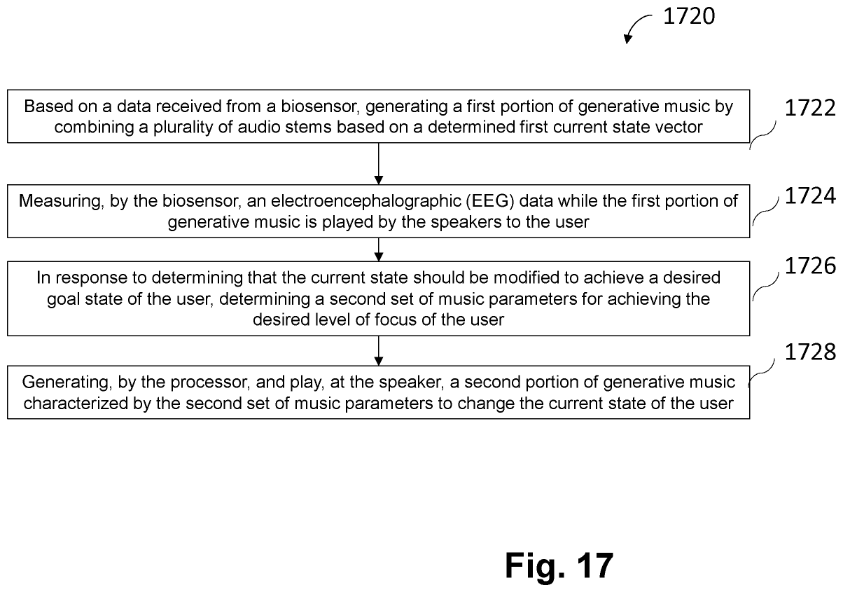

FIG. 17 depicts a flowchart illustrating a method 1720 for generating music for an electronic device, in accordance with at least one embodiment of the present disclosure.

At step 1722, based on a data received from a biosensor, the system 700 generates a first portion of generative music by combining a plurality of audio stems based on a determined first current state vector.

At step 1724, the biosensor measures an electroencephalographic (EEG) data while the first portion of generative music is played by the speakers to the user. At step 1726, in response to determining that the current state should be modified to achieve a desired goal state of the user, the system 700 determines a second set of music parameters for achieving the desired level of focus of the user.

At step 1726 the processor generates a second portion of generative music characterized by the second set of music parameters. The speaker of the system plays the second portion of generative music in order to change the current state of the user.

### EEG Sensor and Headphones

According to a preferred embodiment, the EEG data is collected using an EEG sensor or EEG sensors which is or are appropriate for a context where the user needs to be focused, such as during a significant period of time (a few hours) during which a user performs work of an intellectual nature. Also advantageously, the EEG sensor or sensors should be consistent with the music that is being played to the same user on which EEG data are collected. For this reason, in this embodiment, the EEG sensors can be implemented on a dedicated headset which comprises the EEG sensors installed in an appropriate manner, as well as the headphone speakers for listening to the music.

An example of a suitable device for collecting EEG data as well as playing music can be found in PCT/CA2017/051162, titled “BIOSIGNAL HEADPHONES”, incorporated herein by reference.

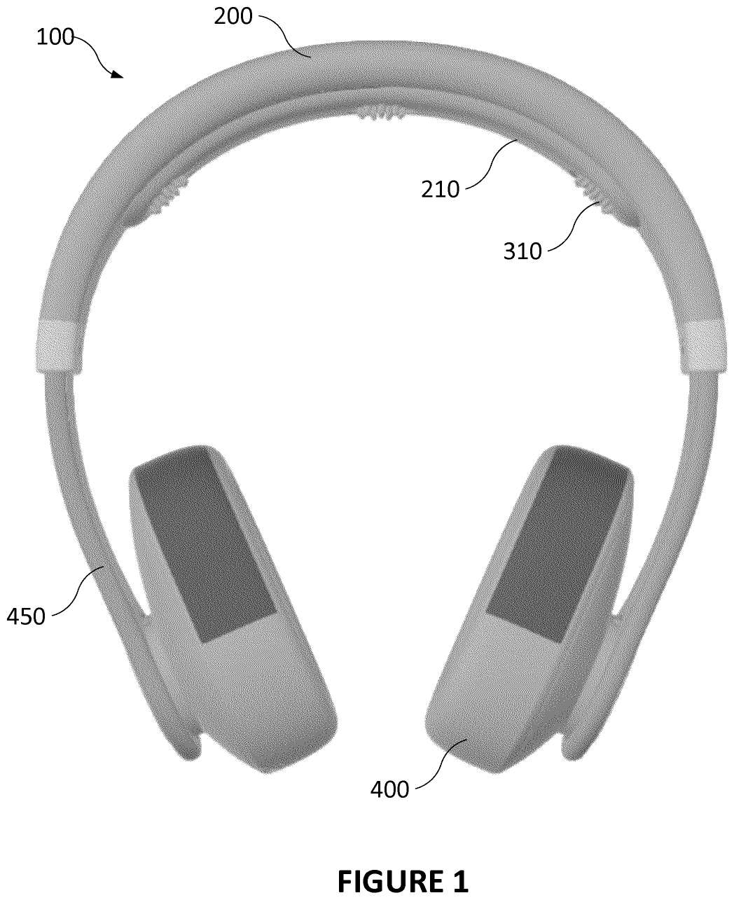

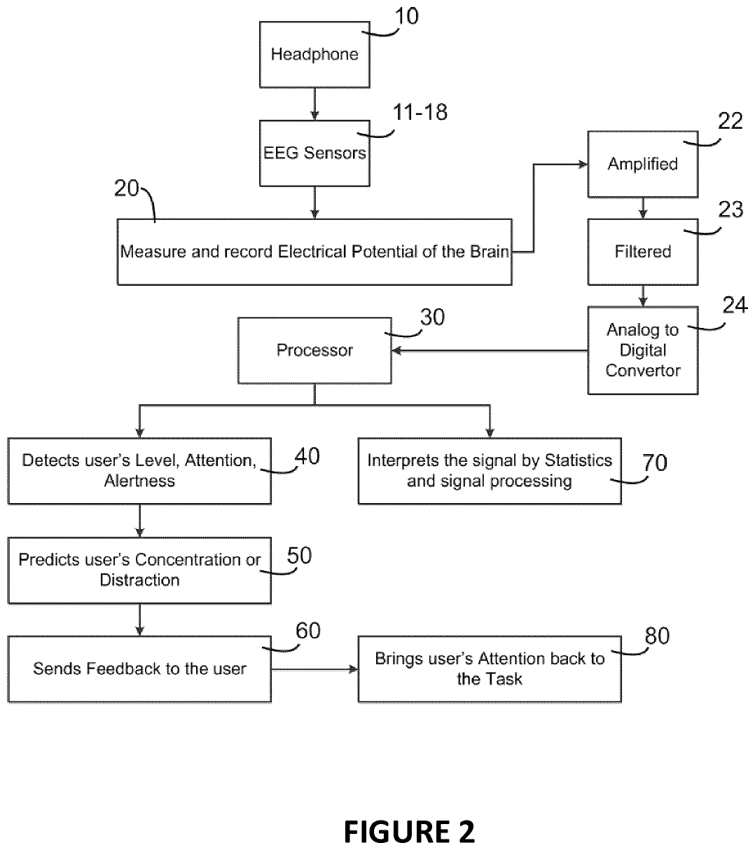

Referring now to FIG. 1, where a first embodiment of the headphone is depicted in accordance with at least one embodiment. As shown in FIGS. 1-2, the headphones 100 uses a plurality of electroencephalographic and biopotential sensors to measure and record electrical potentials originating in the brain. Electrical potentials originating from other sources in the body, such as the heart, the eyes, or muscles, can be measuring by providing sensors at the appropriate locations on the surface of the body. In this case, where electrical potentials originating from the brain are the primary source of data, the sensor electrodes are embedded in an upper band 21 of the headphones 100, measuring (20) voltage on the scalp. This information is processed and relayed to a computer 30, which interprets the signals to determine 40 the current state of the brain. Among other states, the computer detects 40 the user's level of attention and alertness, which are used to predict 50 the user's concentration or distraction with respect to their given task.

The voltage measured by the electrodes is amplified 22, filtered 23, and passed through an analog-to-digital converter 24. According to an embodiment, the signal is then transferred to the computer 30 via Bluetooth, Wi-Fi, or a similar protocol. In the computer 30, the signal is pre-processed in order to remove noise. Several features can then be calculated from the signal, using a variety of statistics and signal processing techniques 70.

According to an embodiment, this information is fed into a machine-learning model, which predicts 50 the state of concentration of the user. This prediction can be used to send feedback 60 to the user of their state of concentration in real-time. The mental state of the user will be actively influenced (based on alarms, reports, etc.) or passively influenced (by subtly changing volume of the music played by the headphone) by this feedback, improving their concentration over time and bring the user's attention back to their task (step 80).

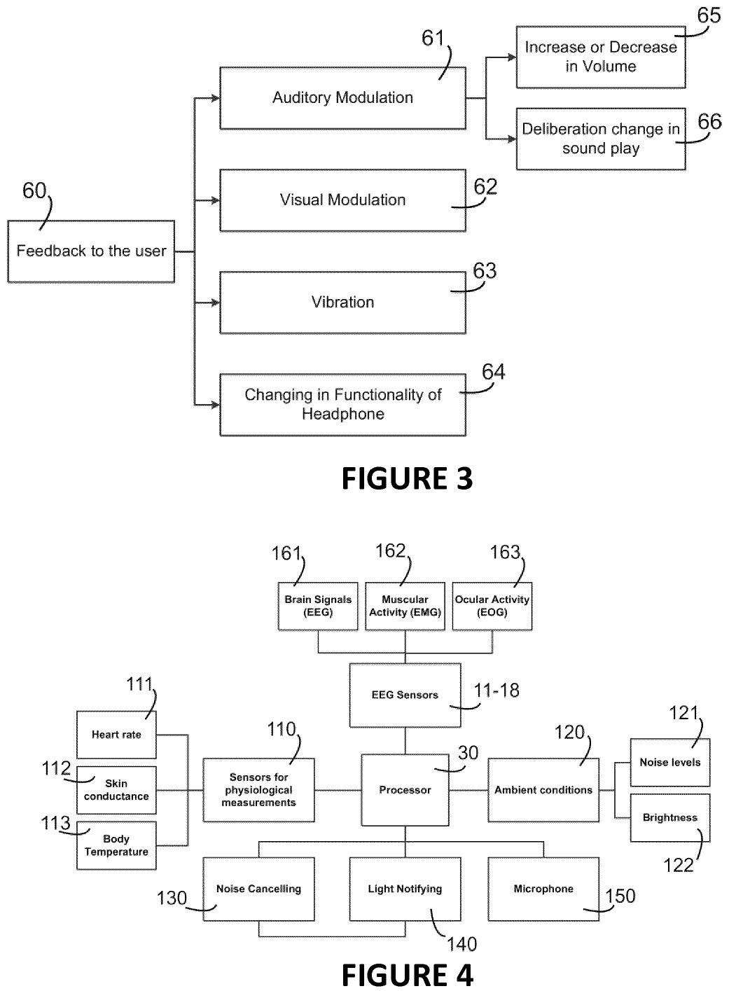

As shown in FIG. 3, the feedback 60 described above can be delivered in the form of a distinguishable notification, the purpose of which is to alert the user of their changed mental state and bring the user's attention back to their task. This will be in the form of an auditory modulation 61—an increase or decrease 65 in the volume, or a deliberate change 66 in the sound played through the headphones. Visual feedback 62 on a computer, mobile device, or integrated light may also be delivered, via modulation of the visuals 62 on the screen. Other forms of feedback include vibration 63, or changes in the functionality of certain headphone features 64 (changing noise cancelling, or turning on/off notifications) or other similar application-level changes. Several forms of feedback may be combined, in order to change the user experience. The feedback may vary in style, intensity, and frequency depending on a variety of user and setting-specific features. The feedback may include the generative music described above.

As shown in FIG. 4, additional sensors embedded in the headphones to detect a variety of physiological measurements 110 including heart rate 111, skin conductance 112, and body temperature 113. Ambient conditions 120 such as noise levels 121 and ambient brightness 122 are also recorded. The computer 30 uses these measurements in addition to the brain activity when predicting attention and alertness, as well as when determining whether to send feedback. The individual combination of sensors and algorithms used in the determination of the user's mental state and in the delivery of feedback will be customized to the user's personal physiology, preferences, daily patterns, and response to previously given feedback.

Again, as shown in FIG. 4, the system comprises electrodes, of passive or active nature, whereas active pertains to the proximity of an amplifier to the source of the signal. The electrodes should be dry electrodes, which are better suited for use with headphones. The electrodes will record brain signals (EEG) 161, muscular activity (EMG) 162, ocular activity (EOG) 163, heart activity (ECG), or any combination of the above.

The headphones 100 are anticipated to be used in a work environment, in order to reduce distraction and improve productivity during a task. The user will be able to customize the feedback experience to the work currently being done. Personal profiles, modulated as a function of the user's preferences and needs, will allow for a catered experience as a function of the desired state.

Using a similar methodology, several other mental or physical states may be predicted via classification of the combination of signals acquired from the headphone's sensors. These may include but are not limited to stress, sadness, anger, hunger, or tiredness. Likewise, the presence of neurological disorders such as epilepsy, anxiety disorder, and attention deficit disorder may be predicted in a similar fashion.

The system may modify human behavior through the delivery of brain-state inspired feedback. These modifications will yield short-term changes in behavior through immediate user response to the feedback provided. An example of this is returning attention to the desired task when notified of the current state of distraction. These modifications can also induce long-term neurophysiological changes due to the user's subconscious response to the feedback provided. An example of this is a subconscious conditioning of the neurological sustained attention system, improving the ability to sustain focus for long durations.

Trends and analytics performed on the recorded bio-signal data provide information on the user's mental and physical state, and allow for prediction of user behavior and their optimal states.

The system uses a combination of one or more sensors to measure bio-signals and ambient conditions, in order to measure and infer the mental and physical state of the user. These sensors include but are not limited to electrodes, temperature probes, accelerometers, pulse oximeters, microphones, and pressure transducers.

The shape and structure of the electrodes are such that they have the capability of passing through the hair and making direct contact with the skin. Examples or embodiments are legged sensors, comb-like structures, flat plates, peg arrays and spring-loaded pegs. The shape and material choice ensure a consistent contact with the skin, minimizing connection impedance.

The system may include a microphone that monitors external ambient noise. This information may be used to modulate the feedback, the music, or the noise cancellation as a function of the level of environmental distraction predicted from the measured ambient conditions. The ambient sound may integrate with the sensor data in order to provide more accurate prediction of the user's mental and physical state. Customizable preferences, including but not limited to the choice of music played through the headphones, may be modulated as a function of the environmental noise. White noise, binaural beats, instrumental music, or user-defined preferences may be used alone or in combination in order to create an ideal work environment for the user. Changes in predicted concentration as a function of the music played may be used to improve focus prediction and feedback delivered.

The system may include passive or active noise isolation. High-density foams, leather, and other materials may be placed around the ear cup in order to isolate the user from external environmental noise. Ambient sound monitoring via the microphone may be used to determine which sounds should be attenuated and which should be amplified.

Body temperature fluctuations may be monitored, and used to improve prediction of the user's mental and physical state. Body temperature may be used to detect long-term trends in user productivity, related to circadian rhythms, energy levels, and alertness. This information may be used to improve the feedback delivered to the user.

Recording of heart rate can provide additional information on body states, including attention and stress levels. Pulse oximetry, balistocardiogram, electrocardiogram, or other substitutable technology may be used for measuring heart rate near the ear or scalp. Analytics performed on heart rate measurements may be used to infer physiological characteristics, including but not limited to heart rate variability, R-R distance, and blood flow volume. These computed physiological characteristics may be used to modulate the feedback delivered to the user, in the form of delivering suggestions for improving concentration.

The system may include sensors in the ear cup, touching the ears or in the area around the ears, for the purpose of recording bio-signals.

The system may include a mechanism for preventing unwanted mechanical movement of the headphones with respect to the head. A possible embodiment of this mechanism is a pad which contacts with the user's head and locks onto the bone structure of the skull, preventing motion of the headphones with respect to the scalp. This mechanism may also be used to promote positioning repeatability of the headphones and sensors on the head.

According to an embodiment, each electrode is embedded in a stabilizing mechanical structure, designed to reduce cable movement, external electrical noise and electrical contact breaks. The stabilizing structure keeps the electrodes in consistent contact with the surface of the user's head during movement.

According to an embodiment, the system comprises an adjustment mechanism, allowing the user to better position the headphones on their head. The mechanism may allow for radial adjustment of the shape of the headphones, adapting for variations in users' head width. The mechanism may allow for adjustable vertical positioning of the sensors, in order to evenly distribute the downward force and ensure proper contact of the electrodes.

Where the system interfaces with the side of the head, leather, fabric, or memory foam may be used for comfort. The material contact interface may be tuned in order to prevent movement of the headphones with respect to the user's head, as well as to dampen vibrations.

Electrodes along the top band may be static, or attached to a moving mechanism that allows the electrodes to retreat completely into the band when not in use. The movement of the electrodes may be controlled via a manually actuated interface, or automatically via the placement of the headphones on the user's head. According to an embodiment, the electrodes are removable, at which point the biosensor headphone becomes a normal headphone. For example, the electrodes can be made removable using a snap-fit connector, or a connector with a male portion engaged in a female portion and held therein with frictional forces.

The system may include a rotational mechanism along the axis connecting the user's ears, allowing the top band to be rotated to contact the forehead, the back of the head, the neck, or other parts of the scalp. This would permit positioning the sensors at other key locations on the head to perform data collection from the prefrontal cortex, the parietal lobe, the occipital lobe, or the neck, for example.

According to an embodiment, the system has the capability of playing an external audio stream over-the-air from a computer or mobile device while simultaneously transferring signals recorded from the headphones to said device. The data-transfer protocol may take place via Bluetooth, Wi-Fi, RF-wave, or other similar wireless protocols.

The system may have an activity light that responds to current brain states. This light notifies other parties of the user's current mental or physical state. One such use is to notify nearby parties that the user is currently busy or concentrated, so as to prevent disturbances.

An alternative embodiment may include the use of this technology as an add-on to existing headphones, connecting to the top band of the headphones and functioning independently of the headphones. An alternative embodiment may also include a multi-purpose band that may be used around the neck, arm, head, leg, or other body part.

The system shall be classified as a computer or computational device, for it not only plays music, but has the capability of recording vital signs and bio-potentials, processing them, and generating an output, independently of whether it is connected to a computer or phone device.

Now referring to FIG. 1, there is shown an embodiment of the headphones 100. The embodiment of the headphones 100 of FIG. 1 comprises a particular design of headband electrodes 310, embedded in a flexible band parallel but distinct from the headband, and earcup electrodes 360.

According to this exemplary embodiment, the headband 200 has a flexible band 210 secured thereto and in which is embedded at least one EEG sensor, or biosensor, i.e., a sensor or electrode measuring electrical activity on the body. According to a preferred embodiment, there are embedded three EEG sensors, or biosensors, in the flexible band 210. Additional EEG sensors can be provided on the earcups 400, e.g., by making a portion of the foam forming the earcup 400 conductive.

As discussed above, typical headbands from usual headphones are not designed to bear EEG sensors. As a result, simply integrating EEG sensors to an existing headphone of a given shape is not likely to offer interesting results in terms of electrical contact between the EEG sensors located thereon and the skin on the person's head, i.e., the scalp.

The embodiment shown in FIG. 1 addresses the issue of suboptimal contact between headphone-mounted EEG sensors and the scalp by providing the EEG sensors on a flexible band distinct (i.e., separate) from the headband and secured to the headband. The flexible band is provided below the headband and is made of a material that renders such band flexible up to the point that the flexible band generally adopts the shape of the head of the user while taking into account that the EEG sensors protrude from the flexible band toward the scalp.

Getting sufficient signals from electrical activity in the brain requires placing electrodes at different locations on the person's head, and not only at the top of the head. In other words, electrodes need to be placed at locations away from the top center of the head, i.e., at more lateral locations on the head. This requirement for electrode placement at more than one location including locations away from the top center (while being within the reach of the headband) creates a strict requirement on the headband shape if one wants to achieve high signal quality and reliability from the sensors at these locations. According to an embodiment, the lateral sensors are distant from the center sensor from about 65 mm (i.e., half the head arc length of a standard person), or between 60 mm and 70 mm, or between 45 mm and 70 mm, or between 45 mm and 80 mm. These distances allow electrodes to lie at the C3 and C4 locations according to the international 10/20 standard.

Prior art headphones with sensors failed to achieve high signal quality and reliability from the sensors at locations away from the top center. Typical headbands for headphones were used for these applications, meaning that the purpose of the headband was solely to mechanically link and electrically connect the earcups, while offering a support, preferably a comfortable one, when being laid on the user's head.

However, as discussed above, the purpose of the headband of the present disclosure, in addition to those of the prior art, is to provide a structure on which the sensors are mounted. These sensors need to be adequately located, maintained at their intended location, and put into contact with the scalp while having a proper contact (to have a high-quality signal) that is maintained over time (so the signal is reliable enough for eventually extract information therefrom).

A flexible band 210, which extends in a shape substantially like a central portion of the headband and is secured under the headband 200 to conform with the user's head when being deformed under the weight of the headphones 100 when being worn.

Each of the headband electrodes is secured at a bottom of the flexible band 210, or lower the headband. The flexible band 210 serves the purpose of adjusting the position of each electrode when the headphones are being worn, such that a contact is maintained with the user's head independently of the position of the headband.

This is done by providing the flexible band 210 with a shape and a material having a flexibility which ensure that upon laying the headband on the user's head, the weight of the headband with the earcups at both ends pushes the flexible band 210 along the surface of the head, including for areas away from the top center of the head. However, the flexible band 210 should keep a rounded shape at rest and in use and simply bend or flex when being used, as it should still have some rigidity (although it should be less rigid or stiff than the upper headband 200). It means that the flexible band 210 should not be confused with a fabric or an elastic band, which would have some drawbacks. Notably, if the flexible band 210 was a fabric or an elastic band, it would not provide proper support for the electrodes, it would not allow them to be easily removable with a snap-fit connector, it would be fragile (i.e., easy to tear), it could expose the inner parts such as cabling, and thus it would not be suited for a consumer product.

The flexible band 210 can be separate from the headband main structure and extending under it. The flexible band is made of any material flexible enough to deform under the weight of the headphone. There are for example many plastics that can deform when a weight corresponding to a few hundred grams is applied on the object. The force is applied by having the central portion of the flexible band 210 applied on the top center of the head and conform therewith, while the lateral portion of the flexible band 210 do not touch the head. If there is no gravity, the flexible band would be at rest, and remain in this position. However, when the headphones 100 are being worn, the gravity pulls down the sides of the flexible band 210 (those closer to the earcups and originally not in contact with the head). These sides of the flexible band 210 are those deformed by gravity and brought down along the surface of the head, to which they conform, at least approximately. The use of a flexible band 210, which has greater flexibility than prior art head bands, and which is closer to the surface of the head, allows a closer and more conforming contact between the flexible band 210 and the head of the user for locations that are more lateral compared to the top center of the head.

The flexible band 210 thus better conforms to the shape of the head than prior art headbands. Electrodes are thus provided in the flexible band 210 and protrude downwardly from the flexible band to reach the scalp of the user. As discussed further below, additional sensors can be placed on or in the earcups. However, the flexible band 210 comprises the sensors that aim at touching the scalp.

According to an embodiment, there are three sensors, one being located at a center of the flexible band 210 in order to be located on the top center of the user head, and two other lateral sensors located away from the center of the flexible band 210, preferably symmetrically from the center, in order to reach lateral locations on the head as discussed above (those for which the presence of the flexible band 210 ensures better and longer-maintained contact).

The flexible band 210 can be sized to ensure that when deformed under the weight of the headphones 100, the flexible band 210 substantially adopts the shape of the surface of the head on which it lies, and has its electrodes protrude at a protruding distance which is consistent with standard hair thickness and is not too short such as to prevent contact with the scalp, or too long which would put all the weight pressure into the legs of the electrodes and thus be uncomfortable. According to an exemplary embodiment, the flexible band 210 has a thickness of about 14 mm, or between 12 mm and 16 mm, or between 10 mm and 18 mm. According to an exemplary embodiment, the flexible band 210 has an arc length of about 196 mm, or between 192 mm and 200 mm, or between 180 mm and 212 mm.

The flexible band 210 is flexible in that it can adopt a variety of radiuses of curvature. The upper headband 200 is more rigid and preferably has a larger radius of curvature, but its radius can change too under the application of forces. According to an exemplary embodiment, the radius of the upper headband 200 can vary from a minimum of about 107 mm to a maximum radius about 136 mm. Other variations and ranges are possible, for example the minimum radius can be in the order of 80 mm to 110 mm, and the maximum radius of curvature can be in the order of 120 mm to 160 mm.

At rest, the flexible band 210 should have a radius of curvature chosen between 80 mm and 100 mm, or preferably between 85 mm and 100 mm, or more preferably between 85 mm and 97 mm, so that the flexible band 210 has a radius of curvature larger than that of most human heads (e.g., 80 percentile), measured at their top area, so as to not conform with a user's head when at rest. Upon being laid on the user's head, the weight of the earcups 400, combined to the force of the top of the end on which the flexible band 210 presses, will force the flexible band to deform. Since it is distinct from the upper headband 200 (although they can look to be together by being housed with an envelope or a protecting fabric), the flexible band will deform so as to conform with the head of the user, thereby adopting a radius of curvature below 85 mm, and preferably below 80 mm, but above 70 mm, as allowed by the resilient material forming the flexible band 210 under the effect of the weight of the headphones (most of it from the earcups and arms) which weighs a few hundred grams (realistically above 100 g and below 1 kg, and more realistically between 150 g and 500 g, and probably between 200 g and 400 g, more probably about 300 g).

When laid on a head, the weight of the earcups 400 pulls down the ends of the flexible band, which transitions from a large radius of curvature to a small radius of curvature, where the large and small radiuses were discussed above.

The headband electrode 310 as used on the flexible band and to be applied onto the scalp of the user. According to an embodiment, the headband sensors 310, or electrodes, comprise a flexible substrate to which legs are attached and protrude downwardly. The flexible substrate can be more flexible than the legs. It means that under the weight of the headphone (which normally has a mass in the order of magnitude of a few hundred grams), when the headband sensor 310 contacts and urges on the user's head, the legs, which are more rigid (or less flexible) than the flexible substrate, will spread (i.e., the rod-shaped leg will change orientation compared to the original orientation which is perpendicular to the flexible substrate) while not particularly changing shape. This spread means that the base of the legs is allowed to change orientation, i.e., that the flexible substrate holding the proximal end of the leg is deformed under such a force to put into effect the independent change of orientation of each one of the legs, for better contact with the scalp. According to an embodiment, the electrode is replaceable by the user.

According to an embodiment, the headphones 100 provides additional sensors, namely earcup sensors on the earcup 400, since collecting data from this region by the ears may be useful in some circumstances. The earcup sensors comprise a conductive material (conductive fabric or polymer, or metal) embedded in the inside of the earcup foam, which can be sewn thereto. The earcup sensor is located at a location on the earcup 400 which allows for making a mechanical (and thus electrical) contact with the back of the user's ear, near the mastoid. The earcup sensor may also comprise a rigid or semi-rigid protrusion on the inside of the earcup 400, which contacts the top or back of the user's ear while the headphones 100 are worn.

The earcup sensors can be provided on a rear surface on at least one earcup 400, i.e., a dual back arrangement, where a first earcup sensor is located at an upper rear location and a second earcup sensor is located at a lower rear location on the inward side of the earcup 400, where they are expected to contact a similarly located area of the rear surface of the ear. Alternatively, there can be provided earcup electrodes on the two sides of at least one of the earcups (back and front, or outward/inward arrangement). This second embodiment covers a greater total surface area but introduces greater complexity as a conductive fabric needs to be sewn on the inward area of the earcups, where it will be in contact with the user head (i.e., the mastoid area), and also exposed to damage. Moreover, outward earcup electrodes can be less performant if the user has hair by the mastoid area, where such an electrode is to be in contact. Inward earcup electrodes are not affected by hair, as there is none on the rear surface of the ear.

The earcup 400 curves around the user's ear (i.e., it is circumaural), maintaining contact with the back of the mastoid. According to an embodiment, the earcup comprises foam. The earcup 400 is smaller than typical prior-art circumaural ear cups (i.e., the type of earcup that surrounds the ear), which typically do not contact the user's ear. It is also larger than typical prior-art on-ear cups, which compress the ear and do not surround it. The earcup 400, according to an embodiment of the present disclosure, thus has a size that would be considered, in the prior art, as an in-between situation which would not be desirable, whereas it is used in the present headphones 100 to ensure proper contact between an inside portion of the earcup and an outside portion of the ear where electrical contact by the sensor 360 may be desirable.

According to an embodiment, the earcup 400 is asymmetric, such that a small lip tucks behind the user's ear when it is being worn. The radius of this lip can be chosen to match the gap between the user's ear and the mastoid, caused by the auriculocephalic angle of the ear. The foam should contact the user's ear primarily at the back of the ear. Contact along the top of the ear is permitted, so long as the applied pressure does not cause discomfort, but is not necessary. The radius of the point of contact between the foam and the ear can be about 5 mm, to ensure that contact is made across a range of ear shapes.

### Low-Level Data Interpretation by the Headset

There is now described an embodiment of a method implemented on a computing system, in communication with the sensors, that performs operations on the signals collected by the sensors to extract meaning information therefrom.

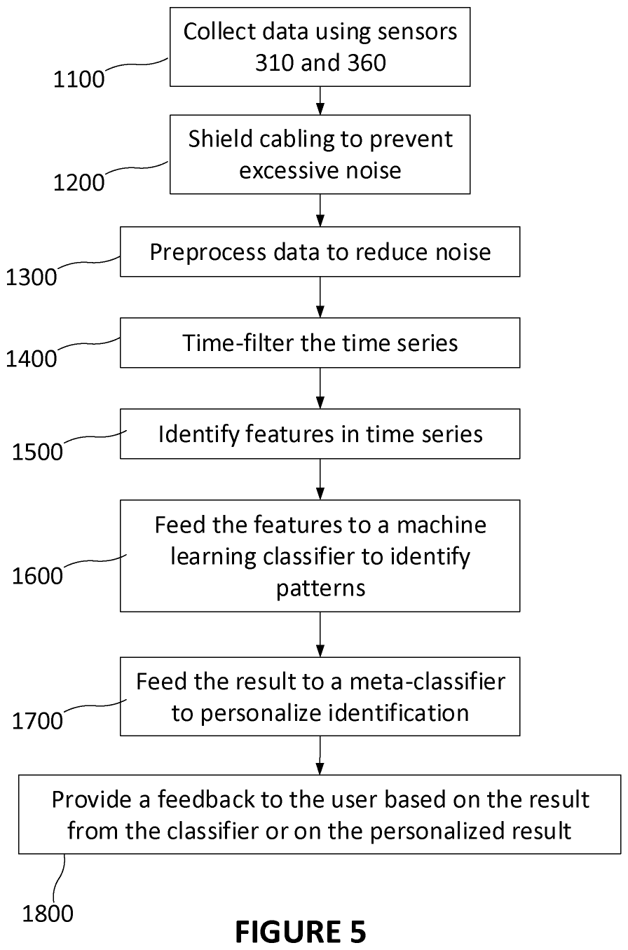

According to an embodiment, and referring to the flowchart of FIG. 5, the data collected by the sensors (step 1100) and routed with the headphones 100 where noise-reduction components are provided (step 1200) are then processed by an embedded processor or sent (preferably wirelessly over a network, or with a wired connection) to a remote computer system in order to implement algorithms for data treatment to extract meaningful information therefrom.

A combination of signal processing, machine learning, and artificial intelligence can be implemented to deliver meaningful results, such as accurate predictions of user concentration from low-dimensional noisy EEG data.

Collected EEG signals are first preprocessed (step 1300). The preprocessing can include, for example, blind source separation algorithms, including PCA, ICA, and wavelet decomposition, and extraction of separable noise sources, including eye blinks and muscle artifacts. According to an embodiment, thresholding is used to identify critical noise sources which are non-separable.

According to an embodiment, the signals are time-filtered (step 1400) using several low and high-order digital FIR and IIR filters to remove high frequency artifacts, low frequency and DC noise sources, powerline noise, and other frequency-based sources of non-EEG noise.

According to an embodiment, the EEG signal, after preprocessing, is separated into features using several signal processing techniques (step 1500). Time-frequency features such as FFT, phase delay, cepstral coefficients, and wavelet transforms can be extracted, for example by applying sliding bins across the time-series data. According to an embodiment, energetic features such as hjorth parameters and zero crossing rate are calculated over windowed bins. Structural information features such as Shannon entropy and Lyapunov exponents are also calculated. These features are measured on each EEG channel, or any linear or nonlinear combination of each channel. The extracted EEG features can be left unprocessed, or can be post-processed using statistical methods, such as smoothing, derivatives, or weighted averaging.

According to an embodiment, in order to describe the state of the person wearing the headphones 100, the features previously identified can be fed into a series of machine learning classifiers (step 1600), which are trained on subsets of the collected data. These classifiers include but are not limited to LDA, SVM, neural networks, decision trees, etc. As a result, each classifier develops the ability to differentiate unique patterns in the EEG signal.

According to an embodiment, these classifiers are fed into a boosted meta-classifier (step 1700), which takes the output of the individual classifiers as inputs. This meta-classifier can be trained on an individual's data, to tailor the classifier system to their unique input and individualize the descriptions or predictions. According to an embodiment, the output of the classifier system is fed into a reinforcement learning model, which determines the likelihood that the user is distracted. The user's state of concentration and distraction is modeled as a Markov decision problem, which the algorithm learns to navigate through use of structures such as Qlearning, and TD difference learning. Feedback (step 1800) is then provided to the user based on the result from the classifier or on the personalized result.

While preferred embodiments have been described above and illustrated in the accompanying drawings, it will be evident to those skilled in the art that modifications may be made without departing from this disclosure. Such modifications are considered as possible variants comprised in the scope of the disclosure.

1. A method for generating music for an electronic device coupled to a server, the method being executable by a processor located on the server, the processor coupled to:
an audio stems database comprising a first plurality of audio stems and a second plurality of audio stems; and
a speaker and a biosensor located in the electronic device, the sensor being configured to measure an electroencephalographic (EEG) data of the user, and the speaker being configured to receive and play a generative music;
the method comprising:
based on comparing of a first current state vector having a first set of musical parameters with stem label vectors of the audio stems, retrieving a first plurality of audio stems from the audio stem database and generating, by the processor, a first portion of generative music by combining the first plurality of audio stems into a plurality of simultaneously played layers, the first portion of generative music having the first set of musical parameters;
measuring, with the biosensor, the EEG data while the first portion of generative music is played by the speakers to the user;
determining, by analyzing the EEG data, a second current state vector that characterizes a second current state of the user;
based on the determined second current state vector, determining whether a current state should be modified to achieve a desired goal state of the user by determining an error state vector;
in response to determining that the current state should be modified, determining a second set of music parameters, for achieving the desired goal state of the user;
based on the second set of music parameters, retrieving a second plurality of audio stems from the audio stem database, and combining the second plurality of audio stems to generate a second portion of generative music characterized by the second set of music parameters; and
transmitting the second portion of generative music to the speaker and collecting, in real time, a second set of EEG data of the user measured while the second portion of generative music is being played to the user by the speakers.

2. The method of claim 1, wherein the processor is coupled to an audio effects database comprising a first plurality of audio effects and wherein generating the first portion of generative music characterized by the first set of musical parameters further comprises:
combining the first plurality of audio stems with the first plurality of audio effects into a plurality of simultaneously played layers.

3. The method of claim 1, wherein determining the first set of musical parameters of the first soundscape is based on a first current state vector.

4. The method of claim 1, wherein determining the second set of musical parameters of the second soundscape is based on a vectorial difference between a goal set of musical parameters and a current set of musical parameters, determined from a vectorial difference between the goal state vector and the second current state vector.

5. The method of claim 1, further comprising determining a current level of focus based on the first current state vector and wherein the desired goal state of the user is a desired level of focus.

6. The method of claim 1, wherein the second set of musical parameters is determined by a machine learning model.

7. The method of claim 5, further comprising:
collecting, in real time, the EEG data of the user to which the second portion of generative music is played and
determining whether the level of focus is improved.

8. The method of claim 2, further comprising:
determining a third set of music parameters of a third portion of the generative music which is more susceptible to force an improvement of a level of focus and
transitioning the generative music automatedly generated into a third portion of generative music based on the third set of music parameters, and
playing the third portion of generative music to the user.

9. The method of claim 1, wherein the server is coupled to another sensor configured to measure environmental data, and the method further comprising receiving environmental data from the electronic device and a context-relevant interaction data indicative of the user interaction with the electronic device and adjusting the second current state vector based on the received environmental data and the context-relevant interaction data.

10. A system for generating music for an electronic device, the system comprising:
an audio stems database comprising a first plurality of audio stems and a second plurality of audio stems; and
a speaker and a biosensor located in the electronic device, the sensor being configured to measure an electroencephalographic (EEG) data of the user, and the speaker being configured to receive and play a generative music; and
a server comprising a processor located on the server,
the processor configured to:
based on comparing of a first current state vector having a first set of musical parameters with stem label vectors of the audio stems, retrieve a first plurality of audio stems from the audio stem database and generate a first portion of generative music by combining the first plurality of audio stems into a plurality of simultaneously played layers, the first portion of generative music having the first set of musical parameters;
receive, from the biosensor, the EEG data while the first portion of generative music is played by the speakers to the user;
determine, by analyzing the EEG data, a second current state vector that characterizes the second current state of the user;
based on the determined second current state vector, determine whether a current state should be modified to achieve a desired goal state of the user by determining an error state vector;
in response to determining that the current state should be modified, determine a second set of music parameters, for achieving the desired goal state of the user;
based on the second set of music parameters, retrieve a second plurality of audio stems from the audio stem database, and combine the second plurality of audio stems to generate a second portion of generative music characterized by the second set of music parameters; and
transmit the second portion of generative music to the speaker and collect, in real time, a second set of EEG data of the user measured while the second portion of generative music is being played to the user by the speakers.

11. The system of claim 10, wherein the processor is coupled to an audio effects database comprising a first plurality of audio effects, and wherein the processor is configured to generate the first portion of generative music characterized by the first set of musical parameters by combining the first plurality of audio stems with the first plurality of audio effects into a plurality of simultaneously played layers.

12. The system of claim 10, wherein the processor is configured to determine the first set of musical parameters of the first soundscape based on the first current state vector.

13. The system of claim 10, wherein determining the second set of musical parameters of the second soundscape is based on a vectorial difference between a goal set of musical parameters and a current set of musical parameters, determined from another vectorial difference between the goal state vector and the second current state vector.

14. The system of claim 10, further comprising determining a current level of focus based on the first current state vector, wherein the desired goal state is a desired level of focus.

15. The system of claim 10, wherein the processor is configured to determine the second set of musical parameters by a machine learning model.

16. The system of claim 14, wherein the server is configured to:
collect, in real time, the EEG data of the user to which the second portion of generative music is played and
determine whether the current level of focus is improved.

17. The system of claim 11, wherein the processor is further configured to
determine a third set of music parameters of a third portion of the generative music which is more susceptible to force an improvement of a level of focus, and
transition the generative music automatedly generated into a third portion of generative music based on the third set of music parameters, and
the system is further configured to play the third portion of generative music to the user generated based on the third set of music parameters.

18. The system of claim 10, further comprising another sensor coupled to the server and configured to measure environmental data, and the processor is further configured to receive environmental data from the electronic device and a context-relevant interaction data indicative of the user interaction with the electronic device and adjusting the second current state vector based on the received environmental data and the context-relevant interaction data.

19. A method for generating music for an electronic device having a biosensor and a speaker and coupled to a processor, the method comprising:
based on biosensor measurement data received from a biosensor, generating a first portion of generative music by combining a plurality of audio stems based on a determined first current state vector;
measuring, by the biosensor, biosensor measurement data while the first portion of generative music is played by the speakers to the user;
in response to determining that the current state should be modified to achieve a desired goal state of the user, determining a second set of music parameters for achieving the desired goal state of the user; and
generating, by the processor, and play, at the speaker, a second portion of generative music characterized by the second set of music parameters to change the current state of the user.

20. The method of claim 19, wherein the biosensor measurement data comprises electroencephalographic (EEG) data.
